(19) United States
(12) Patent Application Publication
(10) Pub. No.: US 2025/0230255 A1
(43) Pub. Date: Jul. 17, 2025

(54) ANTI-CD39 NANOBODY AND USES THEREOF

(71) Applicant: BIOTHEUS INC., Zhuhai (CN)

(72) Inventors: Zhenqing ZHANG, Zhuhai (CN); Yunli JIA, Zhuhai (CN); Yifeng XU, Zhuhai (CN); Xiaoniu MIAO, Zhuhai (CN); Zhiyuan LI, Zhuhai (CN); Andy TSUN, Zhuhai (CN)

(73) Assignee: BIOTHEUS INC., Zhuhai, GD (CN)

(21) Appl. No.: 18/853,158

(22) PCT Filed: Mar. 31, 2023
(86) PCT No.: PCT/CN2023/085567

§ 371 Date: Oct. 1, 2024

(30) Foreign Application Priority Data
Apr. 2, 2022 (CN) 202210345704.7

(51) Int. Cl. C07K 16/28; A61K 39/00; A61P 35/00; G01N 33/573; G01N 33/68

(52) U.S. Cl. CPC C07K 16/2896; A61P 35/00; C07K 16/2818; C07K 16/2827; G01N 33/573; G01N 33/6872; A61K 2039/505; C07K 2317/24; C07K 2317/31; C07K 2317/33; C07K 2317/522; C07K 2317/524; C07K 2317/526; C07K 2317/565; C07K 2317/567; C07K 2317/569; C07K 2317/76; C07K 2317/92; G01N 2333/70596

(57) ABSTRACT

The present invention relates to a nanobody or antigen-binding fragment thereof capable of specifically binding to CD39, a multi-specific antibody comprising the nanobody or antigen-binding fragment thereof, a nucleic acid encoding the nanobody or antigen-binding fragment thereof and a host cell comprising the nucleic acid, as well as relevant use thereof. The invention further relates to the prophylactic, therapeutic, diagnostic and/or detecting use of the nanobody or antigen-binding fragment thereof or the multi-specific antibody.

### TECHNICAL FIELD

The present invention relates to a nanobody or antigen-binding fragment thereof capable of specifically binding to CD39, a multi-specific antibody comprising the same, a nucleic acid encoding the same and a host cell comprising the nucleic acid, as well as relevant use thereof. Further, the present invention relates to the prophylactic, therapeutic, diagnostic and/or detecting use of the nanobody or antigen-binding fragment thereof, or the multi-specific antibody.

### BACKGROUND ART

Human CD39, also known as Ectonucleoside triphosphate diphosphohydrolase 1 (ENTPD1), is a member of ectonucleoside hydrolase, belongs to type II membrane proteins with two transmembrane domains, has a extracellular region in total length of 441 amino acids, and may be present as cleaved form and circulate in the form of soluble CD39 (sCD39). At upstream of adenosine pathway, CD39 hydrolyzes ATP to AMP, AMP is then hydrolyzed by CD73 to adenosine, adenosine acts on adenosine receptor (A2AR) of immune cells, activating downstream protein kinase A (PKA) and CSK kinase, and inhibiting a series of signal pathways related to immune activation, such as LCK, MAPK, PKC (Mosenden et al., 2012), and thereby leads to immunosuppression. Researches have showed that CD39 is highly expressed in various human tumors, including lymphoma, sarcoma, chronic lymphoblastic leukemia, lung cancer, pancreatic cancer, ovarian cancer, kidney cancer, thyroid cancer and testicular cancer. Although in some cases, tumor cells over-express CD39 as compared with normal cells, in the tumor microenvironment, the most consistent cell types showing high CD39 expression include vascular endothelial cells, fibroblasts, and several subsets of immune cells, including NK cells, CD4+CD25+ regulatory T (Treg) cells, macrophages and tumor-specific effector T cells (Li, X. Y. et al, 2019).

In the tumor microenvironment, blocking adenosine mediated immunosuppression by targeting CD39 may inhibit tumor growth, which relates to a mechanism mainly composed of two parts: on one hand, blocking the ATPase activity of CD39 not only reduce adenosine production, but also maintain the ATP level in the tumor microenvironment. ATP can activate dendritic cells (DC cells) and further promote T cell activation by DC cells; on the other hand, CD39 is highly expressed on regulatory T cells and depleted T cells. Blocking the activity of CD39 can reduce the immunosuppressive function of regulatory T cells and reactivate depleted T cells.

However, it was found that, in the tumor microenvironment, CD39 targeted therapy can actually enhance anti-tumor immunity through a variety of mechanisms, including, such as, reducing adenosine mediated T cell immunosuppression, activating inflammasome of macrophages, affecting NK cell function, inhibiting immunosuppressive function of Treg cells, increasing the maturity of antigen presenting cells (APC). In general, these mechanisms act mainly by increasing eATP or reducing adenosine production.

Based on the above mechanisms, several researchers conducted exploratory investigations on the anti-tumor effect of CD39. Preclinical mouse model results showed that antibody targeting CD39 or CD39 gene knockout can effectively arrest tumor growth and metastasis (Jackson et al., 2007). CD39 expressed from both immune cells and non-immune cells can facilitate immune escape, development and metastasis of tumor. In addition, preclinical mouse model results showed that CD39 inhibitor and PD-1 inhibitor could result in good synergistic effect on tumor inhibition.

### SUMMARY OF THE INVENTION

The present inventor has conducted extensive investigations and arrived at nanobodies showing high binding activity to human CD39 and cross-reactivity with cynomolgus CD39. In particular, the nanobody of the invention can effectively alleviate adenosine mediated immunosuppression. In addition, the nanobody are characterized in small molecular weight, superior stability, and others, and thus are advantageous over traditional normal antibodies in terms of drug research and development, such as better tissue permeability, more flexible administration, and easier reconstruction of recombinant proteins.

The invention further provides a multi-specific antibody based on the anti-CD39 nanobody, a composition comprising the nanobody or antigen-binding fragment thereof or the multi-specific antibody, a nucleic acid encoding the nanobody or antigen-binding fragment thereof or the multi-specific antibody, and a host cell comprising the nucleic acid, as well as relevant uses thereof.

### Nanobody or Antigen-Binding Fragment Thereof

Therefore, in the first aspect, the invention provides a nanobody or antigen-binding fragment thereof capable of specifically binding to CD39. The nanobody or antigen-binding fragment thereof comprises:
(a) CDR1, having the sequence shown in SEQ ID NO: 1, or a sequence having substitution, deletion and/or addition of one or more amino acids (e.g., substitution, deletion and/or addition of one, two or three amino acids) as compared with the sequence shown in SEQ ID NO: 1;
(b) CDR2, having the sequence shown in SEQ ID NO: 2, or a sequence having substitution, deletion and/or addition of one or more amino acids (e.g., substitution, deletion and/or addition of one, two or three amino acids) as compared with the sequence shown in SEQ ID NO: 2; and
(c) CDR3, having the sequence shown in SEQ ID NO: 3, or a sequence having substitution, deletion and/or addition of one or more amino acids (e.g., substitution, deletion and/or addition of one, two or three amino acids) as compared with the sequence shown in SEQ ID NO: 3.

In some embodiments, the substitution is a conservative substitution.

In some embodiments, the nanobody or antigen-binding fragment thereof comprises a CDR1 shown in SEQ ID NO: 1, a CDR2 shown in SEQ ID NO: 2, and a CDR3 shown in SEQ ID NO: 3.

In some embodiments, the nanobody or antigen-binding fragment thereof comprises: three CDRs of the VHH as shown in anyone of SEQ ID NOs: 4-8. In some embodiments, the three CDRs of the VHH are determined using Kabat, Chothia or IMGT numbering system.

In some embodiments, the nanobody or antigen-binding fragment thereof comprises an amino acid sequence selected from:
(i) the sequence shown in SEQ ID NO: 4;
(ii) a sequence having substitution, deletion and or addition of one or more amino acids (e.g., substitution, deletion and/or addition of one, two, three, four or five amino acids) as compared with the sequence shown in SEQ ID NO: 4; or
(iii) a sequence having a sequence identity of at least 80%, at least 85%, at least 90%, at least 91%, at least 92%, at least 93%, at least 94%, at least 95%, at least 96%, at least 97%, at least 98%, at least 99%, or 100% with the sequence shown in SEQ ID NO: 4.

In some embodiments, the substitution is a conservative substitution.

In some embodiments, the nanobody or antigen-binding fragment thereof is humanized.

In some embodiments, the nanobody or antigen-binding fragment thereof further comprises a heavy chain framework region of human immunoglobulin (e.g., the heavy chain framework region contained in the amino acid sequence encoded by the human heavy chain embryoid antibody gene), wherein the heavy chain framework region optionally comprises one or more (e.g., 1, 2, 3, 4, 5, 6, 7, 8, 9 or 10) reverse mutations from human residues to camel residues.

In some embodiments, the nanobody or antigen-binding fragment thereof comprises an amino acid sequence selected from:
(i) the sequence shown in any one of SEQ ID NOs: 5-8;
(ii) a sequence having substitution, deletion and or addition of one or more amino acids (e.g., substitution, deletion and/or addition of one, two, three, four or five amino acids) as compared with the sequence shown in any one of SEQ ID NOs: 5-8; or
(iii) a sequence having a sequence identity of at least 80%, at least 85%, at least 90%, at least 91%, at least 92%, at least 93%, at least 94%, at least 95%, at least 96%, at least 97%, at least 98%, at least 99%, or 100% with the sequence shown in any one of SEQ ID NOs: 5-8.

In some embodiments, the substitution is a conservative substitution.

In the second aspect, the invention provides a nanobody or antigen-binding fragment thereof capable of specifically binding to CD39. The nanobody or antigen-binding fragment thereof comprises:
(a) CDR1, having the sequence shown in SEQ ID NO: 9 or 14, or a sequence having substitution, deletion and/or addition of one or more amino acids (e.g., substitution, deletion and/or addition of one, two or three amino acids) as compared with the sequence shown in SEQ ID NO: 9 or 14;
(b) CDR2, having the sequence shown in SEQ ID NO: 10, or a sequence having substitution, deletion and/or addition of one or more amino acids (e.g., substitution, deletion and/or addition of one, two or three amino acids) as compared with the sequence shown in SEQ ID NO: 10; and
(c) CDR3, having the sequence shown in SEQ ID NO: 11 or 15, or a sequence having substitution, deletion and/or addition of one or more amino acids (e.g., substitution, deletion and/or addition of one, two or three amino acids) as compared with the sequence shown in SEQ ID NO: 11 or 15.

In some embodiments, the substitution is a conservative substitution.

In some embodiments, the nanobody or antigen-binding fragment thereof comprises a CDR1 shown in SEQ ID NO: 9 or 14, a CDR2 shown in SEQ ID NO: 10, and a CDR3 shown in SEQ ID NO: 11 or 15.

In some embodiments, the nanobody or antigen-binding fragment thereof comprises:
(1) a CDR1 shown in SEQ ID NO: 9, a CDR2 shown in SEQ ID NO: 10, a CDR3 shown in SEQ ID NO: 11;
(2) a CDR1 shown in SEQ ID NO: 14, a CDR2 shown in SEQ ID NO: 10, a CDR3 shown in SEQ ID NO: 15; or
(3) a CDR1 shown in SEQ ID NO: 14, a CDR2 shown in SEQ ID NO: 10, and a CDR3 shown in SEQ ID NO: 11.

In some embodiments, the nanobody or antigen-binding fragment thereof comprises: three CDRs of the VHH as shown in anyone of SEQ ID NOs: 12, 13 and 16-18. In some embodiments, the three CDRs of the VHH are determined using Kabat, Chothia or IMGT numbering system.

In some embodiments, the nanobody or antigen-binding fragment thereof comprises an amino acid sequence selected from:
(i) the sequence shown in SEQ ID NO: 12;
(ii) a sequence having substitution, deletion and or addition of one or more amino acids (e.g., substitution, deletion and/or addition of one, two, three, four or five amino acids) as compared with the sequence shown in SEQ ID NO: 12; or
(iii) a sequence having a sequence identity of at least 80%, at least 85%, at least 90%, at least 91%, at least 92%, at least 93%, at least 94%, at least 95%, at least 96%, at least 97%, at least 98%, at least 99%, or 100% with the sequence shown in SEQ ID NO: 12.

In some embodiments, the substitution is a conservative substitution.

In some embodiments, the nanobody or antigen-binding fragment thereof is humanized.

In some embodiments, the nanobody or antigen-binding fragment thereof further comprises a heavy chain framework region of human immunoglobulin (e.g., the heavy chain framework region contained in the amino acid sequence encoded by the human heavy chain embryoid antibody gene), wherein the heavy chain framework region optionally comprises one or more (e.g., 1, 2, 3, 4, 5, 6, 7, 8, 9 or 10) reverse mutations from human residues to camel residues.

In some embodiments, the nanobody or antigen-binding fragment thereof comprises an amino acid sequence selected from:
(i) the sequence shown in any one of SEQ ID NOs: 13 and 16-18;
(ii) a sequence having substitution, deletion and or addition of one or more amino acids (e.g., substitution, deletion and/or addition of one, two, three, four or five amino acids) as compared with the sequence shown in any one of SEQ ID NOs: 13 and 16-18; or
(iii) a sequence having a sequence identity of at least 80%, at least 85%, at least 90%, at least 91%, at least 92%, at least 93%, at least 94%, at least 95%, at least 96%, at least 97%, at least 98%, at least 99%, or 100% with sequence shown in any one of SEQ ID NOs: 13, 16-18.

In some embodiments, the substitution is a conservative substitution.

In some embodiments, the CD39 described above in the first or second aspect is selected from human CD39 and or cynomolgus CD39.

In some embodiments, the nanobody or antigen-binding fragment thereof described above in the first or second aspect can block the enzyme activity of CD39 to which it binds.

### Peptide Construction

In the third aspect, the invention further provides a polypeptide construction capable of specifically binding to CD39, comprising the nanobody or antigen-binding fragment thereof described above in the first or second aspect, and an immunoglobulin Fc domain.

As used herein, Fc domain, also known as Fc region, refers to a part of the heavy chain constant region, comprising CH2 and CH3. In some embodiments, the Fc domain comprises a hinge, CH2, and CH3. When the Fc domain comprises a hinge, the hinge regulates the dimerization between the two Fc-containing peptides. The Fc domain may be any isotype of antibody heavy chain constant region. In some embodiments, the Fc domain is IgG1, IgG2, IgG3, or IgG4 Fc region.

In some embodiments, the Fc domain contained in the polypeptide construct of the invention is a natural Fc region, having the same amino acid sequence as those found in nature. For example, the Fc domain may have the same sequence as the natural sequence of human IgG1 Fc region, the natural sequence of human IgG2 Fc region, the natural sequence of human IgG3 Fc region or the natural sequence of human IgG4 Fc region. Natural Fc region may have effector functions. Exemplary “effector functions” include Fc receptor binding; Clq binding and complement dependent cytotoxicity (CDC); antibody dependent cell-mediated cytotoxicity (ADCC); bacteriophage; cell surface receptors (e.g., B cell receptors) down-regulation; and B cell activation. Functional modification can be resulted from the replacement of at least one amino acid residue in the natural Fc region by different residue or chemical modification. As an example, an effector function may be changed (e.g., reduced or enhanced) by changing the affinity of an antibody to an effector ligand (e.g., FcR or complement Clq).

Therefore, in some embodiments, the Fc domain contained in the polypeptide construct of the invention may also be a mutant Fc region, comprising one or more (e.g., 1-10, e.g., 1-5) amino acid mutations or chemical modifications compared with the natural Fc region, to change one or more of the following characteristics of the antibody of the invention: Fc receptor binding, antibody glycosylation, the number of cysteine residues, effector cell function or complement function, etc.

In some embodiments, the Fc domain contained in the polypeptide construct of the invention has ADCC activity. In some embodiments, the Fc domain contained in the polypeptide construct of the invention does not have ADCC activity.

In some embodiments, the immunoglobulin Fc domain is optionally connected to the N-terminal and/or C-terminal (e.g., C-terminal) of the nanobody or antigen-binding fragment thereof through a peptide linker.

In some embodiments, the immunoglobulin Fc domain is a Fc domain of IgG (e.g., the Fc domain of IgG1).

In some embodiments, the immunoglobulin Fc domain comprises the sequence shown in SEQ ID NO: 27, or a sequence having a sequence identity of at least 80%, at least 85%, at least 90%, at least 91%, at least 92%, at least 93%, at least 94%, at least 95%, at least 96%, at least 97%, at least 98%, at least 99%, or 100% with the sequence shown in SEQ ID NO: 27, or a sequence having substitution, deletion and/or addition of one or more amino acids (e.g., substitution, deletion and/or addition of one, two, three, four or five amino acids) as compared with the sequence shown in SEQ ID NO: 27.

### Multi-Specific Antibody

In the fourth aspect, the invention further provides a multi-specific antibody, comprising the nanobody or antigen-binding fragment thereof or the polypeptide construct as described in any of the previous aspects.

In some embodiments, the multi-specific antibody is capable of specifically binding to CD39 and additionally capable of specifically binding to one or more other targets.

In some embodiments, the multi-specific antibody further comprises at least one second antibody having a binding specificity for a second target.

In some embodiments, the multi-specific antibody comprises a nanobody or antigen-binding fragment thereof as described in the first aspect, and at least one second antibody having a binding specificity for a second target.

In some embodiments, the multi-specific antibody comprises a nanobody or antigen-binding fragment thereof as described in the second aspect, and at least one second antibody having a binding specificity for a second target.

In the fifth aspect, the present application provides a multi-specific antibody, comprising a first antigen-binding domain specific to the first epitope of CD39 and a second antigen-binding domain specific to the second epitope of CD39, wherein the first antigen-binding domain comprises the nanobody or antigen-binding fragment thereof as described in the first aspect, and the second antigen-binding domain comprises the nanobody or antigen-binding fragment thereof as described in the second aspect.

In the first exemplary embodiment of the fifth aspect, the first antigen-binding domain and the second antigen-binding domain are both VHH, and the multi-specific antibody comprises a peptide chain II containing a monomeric Fc domain, the first antigen-binding domain and the second antigen-binding domain.

In some embodiments, the monomeric Fc domain comprises CH2 and CH3.

In some embodiments, the multi-specific antibody comprises two peptide chains II. In some embodiments, the monomeric Fc domains of the two peptide chains II form a dimer.

In some embodiments, the two peptide chains II are identical. In some embodiments, the two peptide chains II are different.

In some embodiments, the individual domains are optionally connected through a linker (e.g., a flexible peptide comprising one or more glycine (G) and/or alanine (A)).

It can be readily understood that there is no restriction on relative positions of individual domains in the peptide chain II, as long as their activities can be retained.

In some embodiments, the first antigen-binding domain and the second antigen-binding domain are adjacent and connected therebetween optionally through a linker (e.g., a flexible peptide comprising one or more glycine (G) and or alanine (A)). In some embodiments, the first antigen-binding domain is located at the N-terminal of the second antigen-binding domain. In some embodiments, the first antigen-binding domain is located at the C-terminal of the second antigen-binding domain.

In some embodiments, the peptide chain II comprises, in order of from N terminal to C terminal, adjacent the first antigen-binding domain and the second antigen-binding domain, or adjacent the second antigen-binding domain and the first antigen-binding domain, and further comprises a monomeric Fc domain.

In some embodiments, the peptide chain II comprises, in order of from N terminal to C terminal, the monomeric Fc domain, the first antigen-binding domain and the second antigen-binding domain.

In some embodiments, the first antigen-binding domain is connected to the C-terminal of the monomeric Fc domain through a linker (e.g., a flexible peptide comprising one or more glycine (G) and/or alanine (A)); and or, the second antigen-binding domain is connected to the C-terminal of the first antigen-binding domain through a linker (e.g., a flexible peptide comprising one or more glycine (G) and or alanine (A)).

In some embodiments, the first antigen-binding domain comprises or consists of the amino acid sequence shown in SEQ ID NO: 7; and/or the second antigen-binding domain comprises or consists of the amino acid sequence shown in SEQ ID NO: 16.

In some embodiments, the peptide chain II comprises or consists of the amino acid sequence shown in SEQ ID NO: 21.

In some embodiments, the peptide chain II comprises, in order of from N terminal to C terminal, the second antigen-binding domain, the first antigen-binding domain, and the monomeric Fc domain.

In some embodiments, the first antigen-binding domain is connected to the C-terminal of the second antigen-binding domain optionally through a linker (e.g., a flexible peptide comprising one or more glycine (G) and/or alanine (A)).

In some embodiments, the first antigen-binding domain comprises or consists of the amino acid sequence shown in SEQ ID NO: 7; and/or the second antigen-binding domain comprises or consists of the amino acid sequence shown in SEQ ID NO: 16.

In some embodiments, the peptide chain II comprises or consists of the amino acid sequence shown in SEQ ID NO: 22.

In the second exemplary embodiment of the fifth aspect, the first antigen-binding domain and the second antigen-binding domain are both VHH, and the multi-specific antibody comprises:
(i) a peptide chain I-A comprising the first antigen-binding domain and a light chain constant region (CL); and,
(ii) a peptide chain I-B comprising the second antigen-binding domain and a heavy chain constant region (CH).

In some embodiments, the CL of the peptide chain I-A can form a dimer with the CH1 domain of the heavy chain constant region of the peptide chain I-B.

In some embodiments, the multi-specific antibody comprises two peptide chains I-A and two peptide chains I-B. In some embodiments, the heavy chain constant regions of the two peptide chains I-B form a dimer.

In some embodiments, the two peptide chains I-A are identical. In some embodiments, the two peptide chains I-A are different.

In some embodiments, the two peptide chains I-B are identical. In some embodiments, the two peptide chains I-B are different.

In some embodiments:
(1) the peptide chain I-A comprises, in order of from N terminal to C terminal, the first antigen-binding domain and the light chain constant region (CL); and/or,
(2) the peptide chain I-B comprises, in order of from N terminal to C terminal, the second antigen-binding domain and the heavy chain constant region (CH).

In some embodiments, the first antigen-binding domain comprises or consists of the amino acid sequence shown in SEQ ID NO: 7.

In some embodiments, the peptide chain I-A comprises or consists of the amino acid sequence shown in SEQ ID NO: 20.

In some embodiments, the second antigen-binding domain comprises or consists of the amino acid sequence shown in SEQ ID NO: 16.

In some embodiments, the peptide chain I-B comprises or consists of the amino acid sequence shown in SEQ ID NO: 19.

In the third exemplary embodiment of the fifth aspect, the multi-specific antibody further comprises an antigen-binding domain specific to a target different from CD39.

In some embodiments, the multi-specific antibody further comprises one or more antigen-binding domains that are specific to other targets different from CD39.

In some embodiments, the multi-specific antibody further comprises a third antigen-binding domain that is specific to a target different from CD39.

In the fourth exemplary embodiment of the fifth aspect, the multi-specific antibody further comprises a third antigen-binding domain specific to PD-1.

In some embodiments of the multi-specific antibody, the first antigen-binding domain and the second antigen-binding domain are both VHH, the third antigen-binding domain is a Fab, and the multi-specific antibody comprises:
(1) a peptide chain III-A comprising a light chain variable region of the third antigen-binding domain and a light chain constant region (CL); and
(2) a peptide chain III-B comprising a heavy chain variable region of the third antigen-binding domain, a heavy chain constant region, the first antigen-binding domain, and the second antigen-binding domain.

In some embodiments, the peptide chain III-B comprises, in order of from N terminal to C terminal, adjacent the first antigen-binding domain and the second antigen-binding domain, or adjacent the second antigen-binding domain and the first antigen-binding domain, and further comprises a heavy chain variable region of the third antigen-binding domain and a heavy chain constant region.

In some embodiments, the CL of the peptide chain III-A can form a dimer with the CH1 domain of the heavy chain constant region of the peptide chain III-B.

In some embodiments, the multi-specific antibody comprises two peptide chains III-A and two peptide chains III-B. In some embodiments, the heavy chain constant regions of the two peptide chains III-B form a dimer.

In some embodiments, the two peptide chains III-A are identical. In some embodiments, the two peptide chains III-A are different.

In some embodiments, the two peptide chains III-B are identical. In some embodiments, the two peptide chains III-B are different.

In some embodiments, the individual domains are connected therebetween optionally through a linker (e.g., a flexible peptide comprising one or more glycine (G) and/or alanine (A)).

In some embodiments of the multi-specific antibody:
(1) the peptide chain III-A comprises, in order of from N terminal to C terminal, a light chain variable region of the third antigen-binding domain and a light chain constant region (CL); and/or
(2) the peptide chain III-B comprises, in order of from N terminal to C terminal, a heavy chain variable region of the third antigen-binding domain, a heavy chain constant region, the first antigen-binding domain, and the second antigen-binding domain.

In some embodiments, the multi-specific antibody has one or more of the following characteristics:
(i) the first antigen-binding domain is connected to the C-terminal of the heavy chain constant region through a linker (e.g., a flexible peptide comprising one or more glycine (G) and/or alanine (A)); and/or, the second antigen-binding domain is connected to the C-terminal of the first antigen-binding domain through a linker (e.g., a flexible peptide comprising one or more glycine (G) and/or alanine (A));
(ii) the first antigen-binding domain comprises or consists of the amino acid sequence shown in SEQ ID NO: 7;
(iii) the second antigen-binding domain comprises or consists of the amino acid sequence shown in SEQ ID NO: 16;
(iv) the heavy chain variable region of the third antigen-binding domain comprises VH CDRs1-3 set forth in SEQ ID NOs: 36-38 respectively;
(v) the light chain variable region of the third antigen-binding domain comprises VL CDRs 1-3 set forth in SEQ ID NOs: 39-41 respectively;
(vi) the heavy chain variable region of the third antigen-binding domain comprises or consists of the amino acid sequence shown in SEQ ID NO: 23;
(vii) the light chain variable region of the third antigen-binding domain comprises or consists of the amino acid sequence shown in SEQ ID NO: 24.

In some embodiments, the peptide chain III-A comprises or consists of the amino acid sequence shown in SEQ ID NO: 26.

In some embodiments, the peptide chain III-B comprises or consists of the amino acid sequence shown in SEQ ID NO: 25.

In the sixth aspect, the invention provides a multi-specific antibody capable of specifically binding to both CD39 and PD-1, comprising the nanobody or antigen-binding fragment thereof or the polypeptide construct as described in any of the previous aspects, and an antigen-binding domain specific to PD-1.

In some embodiments, the antigen-binding domain specific to PD-1 comprises VH CDRs 1-3 set forth in SEQ ID NOs: 36-38 respectively, and/or VL CDRs 1-3 set forth in SEQ ID NOs: 39-41 respectively.

In some embodiments, the antigen-binding domain specific to PD-1 comprises a VH shown in SEQ ID NO: 23, and/or a VL shown in SEQ ID NO: 24.

In the seventh aspect, the invention provides an isolated nucleic acid molecule, capable of encoding the nanobody or antigen-binding fragment thereof, the polypeptide construct, or the multi-specific antibody as described in any of the previous aspects.

In some embodiments, the isolated nucleic acid molecule is capable of encoding a nanobody or antigen-binding fragment thereof of the present invention.

In some embodiments, the isolated nucleic acid molecule is capable of encoding a polypeptide construct of the present invention.

In some embodiments, the isolated nucleic acid molecule is capable of encoding a multi-specific antibody of the present invention.

It can be readily understood that the multi-specific antibody of the invention can be composed of one or more polypeptide chains. There is no restriction on the nucleic acid molecular chain number of the isolated nucleic acid molecule encoding the multi-specific antibody of the invention. For example, in some embodiments, the multi-specific antibody is composed of a first peptide chain and a second peptide chain, and the isolated nucleic acid molecule comprises a first nucleotide sequence encoding the first peptide chain and a second nucleotide sequence encoding the second peptide chain, wherein the first nucleoside acid sequence and the second nucleotide sequence exist on the same or different isolated nucleic acid molecules. When the first nucleotide sequence and the second nucleotide sequence exist on different isolated nucleic acid molecules, the isolated nucleic acid molecules in the invention comprise a first nucleic acid molecule comprising the first nucleotide sequence and a second nucleic acid molecule comprising the second nucleotide sequence.

In the eighth aspect, the invention further provides a vector comprising the isolated nucleic acid molecule as described above. In some embodiments, the vector is a cloning vector or an expression vector.

It can be readily understood that the isolated nucleic acid molecule described above can exist in the vector in any form. For example, when the isolated nucleic acid molecule comprises multiple nucleotide sequences encoding different peptide chains, the multiple nucleotide sequences may be located on the same vector or on different vectors. There is no restriction on the orientation, relative position and connection mode of the multiple nucleotide sequences on the vector.

In some embodiments, the vector comprises a first nucleotide sequence encoding the first peptide chain of the multi-specific antibody of the present invention and a second nucleotide sequence encoding the second peptide chain of the multi-specific antibody, wherein the first nucleotide sequence and the second nucleotide sequence exist on the same or different vectors. When the first nucleotide sequence and the second nucleotide sequence exist on different vectors, the vectors of the invention comprise a first vector comprising the first nucleotide sequence and a second vector comprising the second nucleotide sequence.

In the ninth aspect, the present application provides host cell comprising nucleic acid molecules or vectors as described above. Such host cell includes, but is not limited to, prokaryotic cell such as bacterial cell (e.g., E. coli cell), eukaryotic cell such as fungal cell (e.g., yeast cell), insect cell, plant cell and animal cell (e.g., mammalian cell, such as mouse cell, human cell). In some embodiments, the host cell is a microorganism.

The nanobody or antigen-binding fragment thereof, polypeptide construct, or multi-specific antibody of the invention can be prepared by various methods known in the art, such as genetic engineering recombination technology. For example, DNA molecules encoding the nanobody of the invention or antigen-binding fragment thereof, the polypeptide construct, or the multi-specific antibody are obtained by chemical synthesis or PCR amplification. The resulted DNA molecules were inserted into the expression vector and transfected into the host cell. Then, the transfected host cells were cultured under particular conditions to express the nanobody or antigen-binding fragment thereof, the polypeptide construct, or the multi-specific antibody of the present invention.

In the tenth aspect, the present application provides a method for preparing the nanobody or antigen-binding fragment thereof, the polypeptide construct, or the multi-specific antibody as described in any of the previous aspects, comprising culturing the host cell as described above under the condition of allowing protein expression, and recovering the nanobody or antigen-binding fragment thereof or the polypeptide construct or the multi-specific antibody from the cultured host cell culture.

### Preventive Therapeutic Use

In the eleventh aspect, the invention further provides a composition comprising:
(i) the nanobody or antigen-binding fragment thereof as described in the first aspect, a polypeptide construct comprising the nanobody or antigen-binding fragment thereof, a nucleic acid molecule encoding the nanobody or antigen-binding fragment thereof, a vector comprising the nucleic acid molecule, or a host cell comprising the nucleic acid molecule or the vector; and
(ii) the nanobody or antigen-binding fragment thereof as described in the second aspect, a polypeptide construct comprising the nanobody or antigen-binding fragment thereof, a nucleic acid molecule encoding the nanobody or antigen-binding fragment thereof, a vector comprising the nucleic acid molecule, or a host cell comprising the nucleic acid molecule or the vector.

In the twelfth aspect, the present application further provides a pharmaceutical composition, which includes the nanobody or antigen-binding fragment thereof, or the polypeptide construct, or the multi-specific antibody, or the isolated nucleic acid molecule, or the vector, or the host cell, or the composition, as described in any of the previous aspects, as well as pharmaceutically acceptable carriers and/or excipients.

In some exemplary embodiments, the pharmaceutically acceptable carriers and/or excipients comprise sterile injectable liquid (e.g., aqueous or nonaqueous suspension or solution). In some exemplary embodiments, such sterile injectable liquid is selected from water for injection (WFI), bacteriostatic water for injection (BWFI), sodium chloride solution (e.g., 0.9% (w/v) NaCl), glucose solution (e.g., 5% glucose), surfactant-containing solution (e.g., 0.01% Polysorbate 20), pH buffer solution (e.g., phosphate buffer solution), Ringer's solution, and any combination thereof.

In some embodiments, the pharmaceutical composition further comprises an immune checkpoint inhibitor.

In some embodiments, the immune checkpoint inhibitor is selected from anti-PD-1 antibody, anti-PD-L1 antibody, anti-CD73 antibody, or a combination thereof.

In some embodiments, the anti-PD-1 antibody comprises VH CDRs 1-3 set forth in SEQ ID NOs: 36-38 respectively, and/or VL CDRs 1-3 set forth in SEQ ID NOs: 39-41 respectively.

In some embodiments, the anti-PD-1 antibody comprises a VH shown in SEQ ID NO: 23, and/or a VL shown in SEQ ID NO: 24.

In some embodiments, the anti-CD73 antibody comprises a heavy chain shown in SEQ ID NO: 42, and or a light chain shown in SEQ ID NO: 43.

In the thirteenth aspect, the invention further provides the use of the nanobody or antigen-binding fragment thereof, or the polypeptide construct, or the multi-specific antibody, or the isolated nucleic acid molecule, or the vector, or the host cell, or the composition, as described in any of the previous aspects, in the preparation of a medicament for:
(1) reducing the enzyme activity of CD39 in-vitro or in-vivo (e.g., in a human);
(2) alleviating adenosine mediated immunosuppression in a subject (e.g., human);
(3) preventing and/or treating tumors in a subject (e.g., human); or
(4) preventing and/or treating infection in a subject (e.g., human).

In some embodiments, the tumor involves CD39 positive tumor cells.

In some embodiments, the tumor is selected from solid tumor or blood tumor (e.g., leukemia, lymphoma).

In some embodiments, the tumor is selected from colorectal cancer, colon cancer, bladder cancer, breast cancer, uterine/cervical cancer, ovarian cancer, prostate cancer, testicular cancer, esophageal cancer, gastrointestinal cancer, pancreatic cancer, kidney cancer, head and neck cancer, lung cancer, stomach cancer, germ cell cancer, bone cancer, liver cancer, thyroid cancer, skin cancer, tumor of the central nervous system, lymphoma, leukemia, myeloma, sarcoma, and melanoma.

In some embodiments, the tumor is selected from breast cancer, ovarian cancer, testicular cancer, pancreatic cancer, kidney cancer, lung cancer, thyroid cancer, lymphoma, leukemia, myeloma, sarcoma, and melanoma.

In some embodiments, the infection is selected from viral infection, bacterial infection, fungal infection and parasitic infection.

In some embodiments, the subject is a mammal, such as a human or a monkey.

In some embodiments, the nanobody or antigen-binding fragment thereof, or the polypeptide construct, or the multi-specific antibody, or the isolated nucleic acid molecule, or the vector, or the host cell, or the composition is used alone, or in combination with other pharmaceutical active agent(s).

In some embodiments, the nanobody or antigen-binding fragment thereof, or the polypeptide construct, or the multi-specific antibody, or the isolated nucleic acid molecule, or the vector, or the host cell, or the composition is used in combination with other pharmaceutical active agent(s). In some embodiments, the nanobody or antigen-binding fragment thereof, or the polypeptide construct, or the multi-specific antibody, or the isolated nucleic acid molecule, or the vector, or the host cell, or the composition is administered simultaneously with the other pharmaceutical active agent(s). In some embodiments, the nanobody or antigen-binding fragment thereof, or the polypeptide construct, or the multi-specific antibody, or the isolated nucleic acid molecule, or the vector, or the host cell, or the composition is sequentially administered with the other pharmaceutical active agent(s).

In some embodiments, the nanobody or antigen-binding fragment thereof, or the polypeptide construct, or the multi-specific antibody, or the isolated nucleic acid molecule, or the vector, or the host cell, or the composition is used in combination with an immune checkpoint inhibitor. In some embodiments, the nanobody or antigen-binding fragment thereof, or the polypeptide construct, or the multi-specific antibody, or the isolated nucleic acid molecule, or the vector, or the host cell, or the composition is administered simultaneously with the immune checkpoint inhibitor. In some embodiments, the nanobody or antigen-binding fragment thereof, or the polypeptide construct, or the multi-specific antibody, or the isolated nucleic acid molecule, or the vector, or the host cell, or the composition is sequentially administered with the immune checkpoint inhibitor.

In some embodiments, the immune checkpoint inhibitor is selected from anti-PD-1 antibody, anti-PD-L1 antibody, anti-CD73 antibody or anycombination thereof.

In the fourteenth aspect, the present application provides a method for enhancing immune response or preventing and/or treating tumor or infection in a subject, comprising: administering an effective amount of the nanobody or antigen-binding fragment thereof, or the polypeptide construct, or the multi-specific antibody, or the isolated nucleic acid molecule, or the vector, or the host cell, or the composition, or pharmaceutical composition, as described in any of the previous aspects, to subjects in need thereof.

In some embodiments, the tumor involves CD39 positive tumor cells.

In some embodiments, the tumor is selected from solid tumor or blood tumor (e.g., leukemia, lymphoma).

In some embodiments, the tumor is selected from colorectal cancer, colon cancer, bladder cancer, breast cancer, uterine/cervical cancer, ovarian cancer, prostate cancer, testicular cancer, esophageal cancer, gastrointestinal cancer, pancreatic cancer, kidney cancer, head and neck cancer, lung cancer, stomach cancer, germ cell cancer, bone cancer, liver cancer, thyroid cancer, skin cancer, tumor of the central nervous system, lymphoma, leukemia, myeloma, sarcoma, and melanoma.

In some embodiments, the tumor is selected from breast cancer, ovarian cancer, testicular cancer, pancreatic cancer, kidney cancer, lung cancer, thyroid cancer, lymphoma, leukemia, myeloma, sarcoma, and melanoma.

In some embodiments, the infection is selected from viral infection, bacterial infection, fungal infection and parasitic infection.

In some embodiments, the subject is a mammal, such as a human or a monkey.

In some embodiments, the nanobody or antigen-binding fragment thereof, or the polypeptide construct, or the multi-specific antibody, or the isolated nucleic acid molecule, or the vector, or the host cell, or the composition is administered alone, or in combination with the other pharmaceutical active agent(s).

In some embodiments, the nanobody or antigen-binding fragment thereof, or the polypeptide construct, or the multi-specific antibody, or the isolated nucleic acid molecule, or the vector, or the host cell, or the composition is used in combination with other pharmaceutical active agent(s). In some embodiments, the nanobody or antigen-binding fragment thereof, or the polypeptide construct, or the multi-specific antibody, or the isolated nucleic acid molecule, or the vector, or the host cell, or the composition is administered simultaneously with the other pharmaceutical active agent(s). In some embodiments, the nanobody or antigen-binding fragment thereof, or the polypeptide construct, or the multi-specific antibody, or the isolated nucleic acid molecule, or the vector, or the host cell, or the composition is sequentially administered with the other pharmaceutical active agent(s).

In some embodiments, the nanobody or antigen-binding fragment thereof, or the polypeptide construct, or the multi-specific antibody, or the isolated nucleic acid molecule, or the vector, or the host cell, or the composition is administered in combination with the immune checkpoint inhibitor. In some embodiments, the nanobody or antigen-binding fragment thereof, or the polypeptide construct, or the multi-specific antibody, or the isolated nucleic acid molecule, or the vector, or the host cell, or the composition is administered simultaneously with the immune checkpoint inhibitor. In some embodiments, the nanobody or antigen-binding fragment thereof, or the polypeptide construct, or the multi-specific antibody, or the isolated nucleic acid molecule, or the vector, or the host cell, or the composition is sequentially administered with the immune checkpoint inhibitor.

In some embodiments, the immune checkpoint inhibitor is selected from anti-PD-1 antibody, anti-PD-L1 antibody, anti-CD73 antibody, or a combination thereof.

The nanobody or antigen binding fragment thereof, or the polypeptide construct, or the multi-specific antibody, or the pharmaceutical composition of the invention can be formulated into any dosage form known in the medical field, such as tablet, pill, suspension, emulsion, solution, gel, capsule, powder, granule, elixir, troches, suppository, injection (including liquid for injection, sterile powder for injection and concentrated solution for injection), inhalation, and spray forms. The preferred dosage form depends on the expected mode of administration and therapeutic usc. The nanobody or antigen-binding fragment thereof, or the polypeptide construct, or the multi-specific antibody, or the pharmaceutical composition of the invention should be sterile and stable under production and storage conditions. A preferred dosage form is injection. Such injection may be a sterile injection solution. For example, a sterile injection solution may be produced by mixing a necessary amount of the nanobody or antigen-binding fragment thereof, or the polypeptide construct, or the multi-specific antibody, or the pharmaceutical composition of the present invention, into suitable solvent, and optionally together with other desired ingredients (including but not limited to, pH regulator, surfactant, adjuvant, ionic strength enhancer, isotonic agent, preservative, diluent, or any combination thereof), and followed by filtration for sterilization. In addition, the sterile injection solution can be prepared into sterile lyophilized powder (e.g., by vacuum drying or freeze drying) for storage and use later. Such sterile lyophilized powder can be dispersed in suitable vectors before use, such as water for injection (WFI), bacteriostatic water for injection (BWFI), sodium chloride solution (e.g., 0.9% (w/v) NaCl), glucose solution (e.g., 5% glucose), surfactant-containing solution (e.g., 0.01% Polysorbate 20), pH buffer solution (e.g., phosphate buffer solution), Ringer's solution, and any combination thereof.

The nanobody or antigen-binding fragment thereof, or the polypeptide construct, or the multi-specific antibody, or the pharmaceutical composition of the invention can be administered by any suitable means known in the art, including but not limited to oral, buccal, sublingual, periocular, local, parenteral, rectal, intrathecal, intracytoplasmic reticulum, inguinal, bladder, topical (e.g., powder, ointment or drop), or nasal administration. However, for many therapeutic purposes, the preferred administration route/mode is parenteral administration (e.g., intravenous or bolus injection, subcutaneous injection, intraperitoneal injection, intramuscular injection). It should be appreciated that the administration route/mode varies with the intended purpose. In some embodiments, the nanobody or antigen-binding fragment thereof, or the polypeptide construct, or the multi-specific antibody, or the pharmaceutical composition of the present invention is administered by intravenous or bolus injection.

### Test Purpose

In the fifteenth aspect, the present application further provides a conjugate comprising the nanobody or antigen-binding fragment thereof or the polypeptide construct as described in any of the previous aspects, and a detectable marker connected with the nanobody or antigen-binding fragment thereof or the polypeptide construct.

In some embodiments, the detectable marker is selected from an enzyme (e.g., horseradish peroxidase or alkaline phosphatase), a chemiluminescence reagent (e.g., acridine ester compound, luminol and its derivative, or ruthenium derivative), a fluorescent dye (e.g., fluorescein or fluorescent protein), a radionuclide or biotin.

In the sixteenth aspect, the present application further provides a kit comprising the nanobody or antigen-binding fragment thereof or the polypeptide construct or the conjugate as described in any of the previous aspects.

In some embodiments, the kit comprises a conjugate as described above.

In some embodiments, the kit comprises the nanobody or antigen-binding fragment thereof or the polypeptide construct as described in any of the previous aspects, and a second antibody capable of specifically recognizing the nanobody or antigen-binding fragment thereof; optionally, the second antibody optionally further comprises a detectable marker, such as an enzyme (e.g., horseradish peroxidase or alkaline phosphatase), a chemiluminescence reagent (e.g., acridine ester compound, luminol and its derivative, or ruthenium derivative), a fluorescent dye (e.g., fluorescein or fluorescent protein), a radionuclide or biotin.

In the seventeenth aspect, the present application further provides a method for detecting the presence or level of CD39 in a sample, comprising using the nanobody or antigen-binding fragment thereof or the polypeptide construct or the conjugate as described in any one of the previous aspects.

In some embodiments, the method is an immunoassay, such as immunoblotting, enzyme immunoassay (e.g., ELISA), chemiluminescence immunoassay, fluoroimmunoassay or radioimmunoassay.

In some embodiments, the method comprises using a conjugate as described above.

In some embodiments, the method comprises using the nanobody or antigen-binding fragment thereof or the polypeptide construct as described in any one of the previous aspects, and the method further comprises using a second antibody carrying a detectable marker (e.g., an enzyme (e.g., horseradish peroxidase or alkaline phosphatase), a chemiluminescence reagent (e.g., acridine ester compound, luminol and its derivative, or ruthenium derivative), a fluorescent dye (e.g., fluorescein or fluorescent protein), a radionuclide or biotin) for detecting the nanobody or antigen-binding fragment thereof or the polypeptide construct.

In some embodiments, the method comprises: (1) contacting the sample with the nanobody or antigen-binding fragment thereof, the polypeptide construct, or the conjugate of the invention; (2) detecting the formation of antigen-antibody immune complex or determining the amount of the immune complex, wherein the formation of the immune complex indicates the presence of CD39 or CD39-expressing cells.

In the eighteenth aspect, the present application further provides the use of the nanobody or antigen-binding fragment thereof or the polypeptide construct or the conjugate of the invention, as described in any of the previous aspects, in the preparation of a test reagent for detecting the presence or level of CD39 in a sample.

In some embodiments, the test reagent detects the presence or level of CD39 in the sample by the method described above for detecting the presence or level of CD39 in a sample.

In some embodiments, the sample is a cell sample (e.g., tumor cell) from a subject (e.g., a mammal, preferably a human or monkey).

### Definition of Terms

In the present invention, unless otherwise stated, the scientific and technical terms used herein have the meanings commonly understood by a person skilled in the art. In addition, the virology, biochemistry and immunology laboratory operations used herein are all routine operations generally used in corresponding fields. Nevertheless, for easy understanding of the invention, the definitions and explanations of relevant terms are provided below.

When the terms “for example”, “such as”, “e.g.”, “comprise”, “include” or variations thereof are used herein, these terms should not be considered restrictive, but should be interpreted as open-ended, i.e., indicating “but not limited to” or “not limited to”.

Unless otherwise specified herein or obviously contradictory in the context, the terms “a/an” and “one” and “the” and similar wording, used in the context of the invention (especially, in the context of appended claims), shall be interpreted as encompassing both single and multiple referents.

As used herein, the term “nanobody” has the meaning commonly understood by a person skilled in the art and refers to an antibody fragment composed of a single monomeric variable antibody domain (e.g., a single heavy chain variable region), usually derived from the variable region of a heavy chain antibody (e.g., a camelidae antibody or shark antibody). Typically, the nanobody is composed of four framework regions and three complementary determinant regions, in the structure of FR1-CDR1-FR2-CDR2-FR3-CDR3-FR4. A nanobody may be truncated at the N-terminal or C-terminal, with part of FR1 and/or FR4 removed or one or two of the framework regions deleted, as long as the antigen binding capability and specificity are substantially retained. Nanobody is also called single-domain antibody (sdAb), the two are used interchangeably herein.

As used herein, the term “antigen-binding fragment” of a nanobody refers to a polypeptide comprising a segment of the nanobody and maintaining the ability of specifically binding the same antigen as that the nanobody binds and/or competing with the nanobody for specific binding to antigen, also called “antigen-binding portion”. Generally, see, Fundamental Immunology, Ch. 7 (Paul, W., cd., 2nd edition, Raven Press, N.Y. (1989)), incorporated herein by reference in its entirety and for all purposes. The antigen-binding fragment of the nanobody of the invention can be produced by recombinant DNA technology, or by enzymatic or chemical cleavage of the nanobody of the invention. In some embodiments, the “antigen-binding fragment” of the nanobody may be truncated at the N-end or C-terminal, as compared with the full-length nanobody, with part of FR1 and/or FR4 removed or one or two of those framework regions deleted, as long as the antigen-binding capability and specificity are substantially retained.

An antigen-binding fragment of a nanobody can be obtained from a given nanobody (e.g., the nanobody provided by the invention) using conventional techniques known to a person skilled in the art (e.g., recombinant DNA technology, or enzymatic or chemical cleavage method), and the resulting antigen-binding fragment may be screened for specificity in the same way as for the complete nanobody.

As used herein, unless the context clearly indicates, when reference was made to the term “nanobody”, it comprises not only the complete nanobody, but also the antigen-binding fragment of the nanobody.

As used herein, the term “multi-specific antibody” refers to an antibody that has binding specificity for at least two (e.g., two, three or four) different antigens (or epitopes). A multi-specific antibody comprises multiple antigen-binding domains with binding specificity for different antigens (or epitopes), so that it can bind to at least two different binding sites and/or target molecules. Each antigen-binding domain contained in the multi-specific antibody can be independently selected from a full-length antibody (e.g., IgG antibody) or its antigen-binding fragment (e.g., Fv fragment, Fab fragment, F(ab′)₂ fragment or scFv). In some cases, the antigen-binding domains are connected therebetween by peptide linker.

As used herein, the term “complementary determining region” or “CDR” refers to the amino acid residues responsible for antigen binding in the variable region of the antibody. There are three CDRs, named CDR1, CDR2 and CDR3, in a nanobody. The precise boundaries of these CDRs can be defined according to various numbering systems known in the art, such as the Kabat numbering system (Kabat et al., Sequences of Proteins of Immunological Interest, 5th Ed. Public Health Service, National Institutes of Health, Bethesda, Md., 1991), the Chothia numbering system (Chothia&Lesk (1987) J. Mol. Biol. 196:901-917; Chothia et al. (1989) Nature 342:878-883) or IMGT numbering system (Lefranc et al., Dev. Comparat. Immunol. 27:55-77, 2003). For a given nanobody, a person skilled in the art will easily identify its CDR defined by each numbering system. Moreover, the correspondences among different numbering systems are familiar to a person skilled in the art (e.g., see Lefranc et al., Dev. Comparat. Immunol. 27:55-77, 2003). As preferably used herein, the CDRs of the nanobody is defined by Kabat numbering system.

As used herein, the term “framework region” or “FR” residue refers to the amino acid residues other than the CDR residues defined above, in the variable region of the antibody.

As used herein, the term “Fab fragment” or “Fab” means an antibody fragment composed of a light chain fragment comprising VL and CL and a heavy chain fragment comprising VH and CH1.

As used herein, the term “Fc domain” or “Fc region” means a part of the heavy chain constant region comprising CH2 and CH3. Fc fragment of antibody has several different functions but does not participate in antigen binding. The “effector function” mediated by Fc region comprises Fc receptor binding; Clq binding and complement dependent cytotoxicity (CDC); antibody dependent cell-mediated cytotoxicity (ADCC); bacteriophage; cell surface receptors (e.g., B cell receptors) down-regulation; and B cell activation. In some embodiments, the Fc region comprises hinge, CH2 and CH3. When the Fc region comprises a hinge, the hinge regulates the dimerization between the two Fc-containing peptides. The Fc region may be any isotype of antibody heavy chain constant region, such as IgG1, IgG2, IgG3 or IgG4.

Fc domain may be a natural Fc region or a variant Fc region. A natural Fc region comprises an amino acid sequence consistent with the amino acid sequence of the Fe region found in nature. For example, a natural sequence of human Fc region comprises the natural sequence of human IgG1 Fc region (both non-A isotype and A isotype); the natural sequence of human IgG2 Fc region; the natural sequence of human IgG3 Fc region; the natural sequence of human IgG4 Fc region, as well as their naturally occurring variants. A variant Fc region comprises an amino acid sequence different from the natural sequence of Fc region resulted from at least one amino acid modification. In some embodiments, a variant Fc region may have changed effector function (e.g., Fc receptor binding, antibody glycosylation, the number of cysteine residues, effector cell function or complement function) different from the natural Fc region.

As used herein, the term “humanization” refers to the modification of non-human antibody by genetic engineering, so that its amino acid sequence is modified to improve the homology with the sequence of human antibody. Generally, all or part of the CDR regions of a humanized antibody are derived from non-human antibody (donor antibody), and all or part of non-CDR regions (e.g., variable region FR and/or constant region) are derived from human immunoglobulin (receptor antibody). In some embodiments, a humanized antibody has its CDR region derived from non-human antibody (donor antibody), and all or part of its non-CDR regions (e.g., variable region FR and/or constant region) derived from human immunoglobulin (receptor antibody). A humanized antibody usually retains the expected properties of the donor antibody, including but not limited to antigen specificity, affinity, reactivity, etc. As used herein, the donor antibody can be a camelidae antibody with expected properties (e.g., antigen specificity, affinity, reactivity, etc.). For preparing humanized antibodies, the CDR region derived from an immunized animal is inserted into the human framework sequence using methods known in the art. In the context of nanobody, humanized antibody may refer to humanized VHH, i.e., a VHH in which one or more framework regions have been replaced by corresponding human framework regions. In some cases, some framework regions (FR) of human immunoglobulin are replaced by corresponding non-human residues. In addition, a humanized VHH may comprise residues that are not found in the initial VHH or human framework sequence but are included to further improve and optimize the performance of the VHH or the VHH-containing peptide.

As used herein, the term “identity” refers to the matching of sequences between two polypeptides or between two nucleic acids. In the determination of the percentage identity of two amino acid sequences or two nucleic acid sequences, the sequences are aligned for the best comparison purpose (e.g., a gap may be introduced in the first amino acid sequence or nucleic acid sequence for best matching the second amino acid or nucleic acid sequence). Then the amino acid residues or nucleotides at the corresponding amino acid position or nucleotide position are compared. When a position in the first sequence is occupied by the same amino acid residue or nucleotide as the corresponding position in the second sequence, the two molecules are identical at that position. The percentage identity between two sequences is a function of the number of identical positions shared by the sequences (i.e., percentage identity=number of identical overlap positions/total number of positions× 100%). In some embodiments, the two sequences have the same length.

The determination of the percentage identity between two sequences can also be achieved by using mathematical algorithms. A non-limiting example of a mathematical algorithm for comparing two sequences is the algorithm of Karlin and Altschul (1990, Proc Natl. Acad. Sci. U.S.A. 87:2264-2268), as well as modified by Karlin and Altschul (1993, Proc Natl. Acad. Sci. U.S.A. 90:5873-5877) by integrating the algorithm into the NBLAST and XBLAST programs of Altschul et al. (1990, J. Mol. Biol. 215:403).

As used herein, the term “specifically binding” refers to a non-random binding reaction between two molecules, such as the reaction between an antibody and its targeted antigen. The strength or affinity of the specific binding interaction can be expressed by the equilibrium dissociation constant (K_{D}) of the interaction. As used herein, the term “K_{D}” refers to the dissociation equilibrium constant of a particular antibody-antigen interaction, describing the binding affinity between antibody and antigen. A smaller equilibrium dissociation constant means a closer antibody-antigen binding, and a higher affinity between antibody and antigen.

The specific binding property between the two molecules can be determined using methods known in the art. One method involves measuring the formation and dissociation rates of antigen binding sites/antigen complexes. Both the “binding rate constant” (ka or kon) and the “dissociation rate constant” (kdis or koff) can be calculated from the concentration and the actual association and dissociation rates (see Malmqvist M, Nature, 1993, 361:186-187). The ratio of kdis/kon is equal to the dissociation constant K_{D} (see Davies et al., Annual Rev Biochem, 1990, 59:439-473). K_{D}), kon and kdis values may be measured by any practical method. In some embodiments, surface plasmon resonance (SPR) may be used in Biacore for determining the dissociation constant. In addition, bioluminescence interferometry or Kinexa may also be used for determining the dissociation constant.

As used herein, the term “vector” refers to a nucleic acid carrier with polynucleotides inserted therein. When a vector expresses the protein encoded by the inserted polynucleotide, it is called expression vector. A vector may be transferred into host cell through transformation, transduction or transfection, so that the genetic material elements it carries can be expressed in the host cell. Vector is well known to a person skilled in the art, including but not limited to: plasmid; bacteriophage; Cox plasmid; artificial chromosome, such as yeast artificial chromosome (YAC), bacterial artificial chromosome (BAC) or artificial chromosome (PAC) from P1 source; phages such as λ phage or M13 phage and animal virus, etc. Animal viruses that can be used as vector include, but are not limited to, retrovirus (including lentivirus), adenovirus, adeno-associated virus, herpes virus (e.g., herpes simplex virus), poxvirus, baculovirus, papillomavirus, and papillomavirus (e.g., SV40). A vector can comprise a variety of elements that control expression, including but not limited to promoter sequence, transcription initiation sequence, enhancer sequence, selection element and reporter gene. In addition, the vector can further comprise a replication starting site.

As used herein, the term “host cell” refers to the cells into which the vector is introduced, including but not limited to prokaryotic cells such as E. coli or Bacillus subtilis, fungal cells such as yeast cells or aspergillus, insect cells such as S2 drosophila cells or Sf9, or animal cells such as fibroblasts; CHO cells; COS cells; NSO cells; HeLa cells; BHK cells; HEK 293 cells; or human cells.

As used herein, the term “conservative substitution” means an amino acid substitution that does not adversely affect or change the expected properties of the protein/peptide comprising the amino acid sequence. For example, a conservative substitution may be introduced through standard techniques known in the art, such as site-specific mutagenesis and PCR-mediated mutagenesis. Conservative amino acid substitution comprises the replacement of an amino acid residue by another amino acid residue having similar side chain, such as replaced by a residue that is physically or functionally similar to the corresponding amino acid residue (e.g., having similar size, shape, charge, chemical properties including the ability to form covalent bonds or hydrogen bonds, etc.). A group list of amino acid residue with similar side chains has been defined in the art including those amino acids with basic side chains (e.g., lysine, arginine and histidine), acidic side chains (e.g., aspartic acid and glutamic acid), uncharged polar side chains (e.g., glycine, asparagine, glutamine, serine, threonine, tyrosine, cysteine, tryptophan), non-polar side chains (e.g., alanine, valine, leucine, isoleucine, proline, phenylalanine, methionine), β branched side chains (e.g., threonine, valine, isoleucine), and aromatic side chains (e.g., tyrosine, phenylalanine, tryptophan, histidine). Therefore, corresponding amino acid residue is preferably replaced by another amino acid residue from the same group. The method for identifying conservative substitution of amino acid is well known in the art (see, e.g., Brummell et al., Biochem. 32:1180-1187 (1993); Kobayashi et al., Protein Eng. 12 (10): 879-884 (1999); and Burks et al., Proc Natl Acad. Set USA 94:412-417 (1997), all are incorporated herein by reference).

With regard to the twenty conventional amino acids involved herein, they are written following the conventional usage. See, e.g., Immunology-A Synthesis (2nd Edition, E. S. Golub and D. R. Green, Eds., Sinauer Associates, Sunderland, Mass. (1991)), which is incorporated herein by reference. As used herein, the terms “polypeptide” and “protein” have the same meaning and can be used interchangeably. Moreover, as used herein, amino acids are usually represented by single-letter and three-letter abbreviations known in the art. For example, alanine can be expressed as A or Ala.

As used herein, the term “pharmaceutically acceptable carrier and/or excipient” means those carriers and/or excipients pharmacologically and/or physiologically compatible with the subject and the active ingredient, as well-known in the art (see, e.g., Remington's Pharmaceutical Sciences. Edited by Gennaro A R, 19th ed. Pennsylvania: Mack Publishing Company, 1995), and comprises but is not limited to: pH regulator, surfactant, adjuvant, ion strength enhancer, diluent, osmotic pressure maintaining reagent, absorption-delaying reagent, and preservative. For example, pH regulators include but are not limited to phosphate buffer. Surfactants include but are not limited to cationic, anionic or non-ionic surfactants, such as Tween-80. Ion strength enhancers include but are not limited to sodium chloride. The reagents for maintaining osmotic pressure include but are not limited to sugar, NaCl, and analogues thereof. The absorption-delaying reagents include but are not limited to monostearate and gelatin. Diluents include but are not limited to water, aqueous buffer solution (e.g., buffered saline), alcohol and polyol (e.g., glycerin), etc. Preservatives include but are not limited to various anti-bacterial and anti-fungal agents, such as thiomersal, 2-phenoxyethanol, p-hydroxybenzoate, tert-butyl trichloride, phenol, sorbic acid, etc. Stabilizer has the meaning commonly understood by a person skilled in the art, i.e., capable of stabilizing the expected activity of pharmaceutical active ingredient, including but not limited to sodium glutamate, gelatin, SPGA, saccharides (e.g., sorbitol, mannitol, starch, sucrose, lactose, glucan, or glucose), amino acids (e.g., glutamic acid, glycine), protein (e.g., dried whey, albumin or casein) or its degradation products (e.g., lactoalbumin hydrolysatc), etc. In some exemplary embodiments, the pharmaceutically acceptable carrier or excipient comprises a sterile injectable liquid (e.g., an aqueous or nonaqueous suspension or solution). In some exemplary embodiments, such sterile injectable liquid is selected from water for injection (WFI), bacteriostatic water for injection (BWFI), sodium chloride solution (e.g., 0.9% (w/v) NaCl), glucose solution (e.g., 5% glucose), surfactant-containing solution (e.g., 0.01% Polysorbate 20), pH buffer solution (e.g., phosphate buffer solution), Ringer's solution, and any combination thereof.

### Beneficial Effects of the Invention

The invention provides a nanobody showing high binding activity to human CD39 and cross-reactivity with cynomolgus CD39. In particular, the nanobody of the invention can effectively alleviate adenosine mediated immunosuppression. In addition, the nanobody are characterized in small molecular weight, superior stability, and others, and thus are advantageous over traditional normal antibodies in terms of drug research and development, such as better tissue permeability, more flexible administration, and easier reconstruction of recombinant proteins.

In addition, the invention further provides a multi-specific antibody based on the anti-CD39 nanobody, which, when administered in an animal model, significantly inhibits the tumor growth and thus has significant clinical value.

The embodiments of the invention are described in detail below in combination with the drawings and examples. However, it should be appreciated that the following drawings and examples are only illustrative, but not limiting. On basis of the following detailed description of the drawings and preferred embodiments, various purposes and advantageous aspects of the present invention will become apparent to a person skilled in the art.

### BRIEF DESCRIPTION OF THE DRAWINGS

FIG. 1 shows the test results of binding activity of anti-CD39 nanobodies to CHO-hCD39 cell.

FIG. 2 shows the test results of binding activity of anti-CD39 nanobodies to CHO-cyCD39 cell.

FIG. 3 shows the test results of anti-CD39 nanobodies binding epitope Bin.

FIG. 4 shows the test results of anti-CD39 nanobodies for blocking CD39 enzyme activity in human CD39 overexpression cells.

FIG. 5 shows the test results of anti-CD39 nanobodies for blocking CD39 enzyme activity in PBMC system.

FIG. 6 shows the test results of anti-CD39 nanobodies for blocking the enzyme activity of soluble CD39.

FIG. 7 shows affinity test results of humanized nanobodies to CHO-hCD39 cells.

FIG. 8 shows affinity test results of humanized nanobodies to CHO-cyCD39 cells.

FIG. 9 shows the test results of humanized nanobodies for blocking CD39 enzyme activity in human CD39 overexpression cells.

FIG. 10 shows the test results of humanized nanobodies for blocking CD39 enzyme activity in PBMC system.

FIG. 11 shows the test results of humanized nanobodies for blocking the enzyme activity of soluble CD39.

FIG. 12 shows the test results of humanized nanobodies for blocking CD39 enzyme activity in human CD39 overexpression cells.

FIG. 13 shows the test results of humanized nanobodies for blocking CD39 enzyme activity in PBMC system.

FIG. 14 shows the test results of humanized nanobodies for blocking the enzyme activity of soluble CD39.

FIG. 15 shows the test results of humanized nanobody reversing CD4+T cell proliferation inhibition.

FIG. 16 shows the test results of humanized nanobody reversing CD8+T cell proliferation inhibition.

FIG. 17 shows the structural designs of bi-epitopic antibodies (Bi307/308 and Fc-37-46).

FIG. 18 shows the test results of bi-epitopic antibodies (Bi307/308 and Fc-37-46) for blocking CD39 enzyme activity in human CD39 overexpression cells.

FIG. 19 shows the test results of bi-epitopic antibodies (Bi307/308 and Fc-37-46) for blocking the enzyme activity of the soluble CD39.

FIG. 20 shows the test results of bi-epitopic antibodies (Bi307/308 and Fc-37-46) for blocking CD39 enzyme activity in PBMC system.

FIG. 21 shows the PK test results of bi-epitopic antibodies (Bi307/308 and Fc-37-46) in mice.

FIG. 22 shows the pharmacodynamic test results of bi-epitopic antibodies (Bi307/308 and Fc-37-46) in tumor-bearing mice inoculated with MDA-MB-231 cell overexpressing hCD39.

FIG. 23 shows the pharmacodynamic test results of bi-epitopic antibodies (Bi307/308 and Fc-37-46) in Molp-8 tumor model mice.

FIG. 24 shows the structural design of the bi-epitopic antibody (46-37-Fc) with N-terminal based structure.

FIG. 25 shows the test results of the bi-epitopic antibodies for blocking the enzyme activity in MOLP-8 tumor cells.

FIG. 26 shows the test results of the bi-epitopic antibodies for blocking the enzyme activity of soluble CD39.

FIG. 27 shows the test results of bi-epitopic antibodies for blocking CD39 enzyme activity in PBMC system.

FIG. 28 shows the test results of the bi-epitopic antibodies reversing T cell proliferation inhibition.

FIG. 29 shows the PK test results of bi-epitopic antibody (Fc-37-46) in mice.

FIG. 30 shows the pharmacodynamic test results of bi-epitopic antibodies in tumor-bearing mice inoculated with A375 cell overexpressing hCD39.

FIG. 31 shows the structural design of anti-PD1×CD39 antibody.

FIG. 32 shows the test results of binding activity of anti-PD1×CD39 antibody to CHO-hCD39 cell.

FIG. 33 shows the test results of binding activity of anti-PD1×CD39 antibody to CHO-cynoCD39 cell.

FIG. 34 shows the test results of binding activity of anti-PD1×CD39 antibody to CHO-hPD1 cell.

FIG. 35 shows the test results of binding activity of anti-PD1×CD39 antibody to CHO-cynoPD1 cell.

FIG. 36 shows the test results of anti-PD1×CD39 antibody for blocking the enzyme activity in MOLP-8 tumor cells.

FIG. 37 shows the test results of anti-PD1×CD39 antibody for blocking CD39 enzyme activity in PBMC.

FIG. 38 shows the blocking activity test result of anti-PD1×CD39 antibody for blocking PD1/PD-L1 binding.

FIG. 39 shows the pharmacodynamic test results of anti-PD1×CD39 antibody in tumor-bearing mice inoculated with A375 cell overexpressing hCD39.

### SEQUENCE INFORMATION

The sequences involved in the invention are described in the following table.

TABLE 1
Sequence information

| SEQ | |
|---|---|
| ID | |
| NO: | Sequence description |
| 1 | R-Ye-19(1)-037/HZ-R-Ye-19(1)-037-1/HZ-R-Ye- |
| | 19(1)-037-2/HZ-R-Ye-19(1)-037-3/HZ-R-Ye- |
| | 19(1)-037-4 CDR1 amino acid sequence EYSMG |
| 2 | R-Ye-19(1)-037/HZ-R-Ye-19(1)-037-1/HZ-R-Ye- |
| | 19(1)-037-2/HZ-R-Ye-19(1)-037-3/HZ-R-Ye- |
| | 19(1)-037-4 CDR2 amino acid sequence AISGFG |
| | HITHYADSVKG |
| 3 | R-Ye-19(1)-037/HZ-R-Ye-19(1)-037-1/HZ-R-Ye- |
| | 19(1)-037-2/HZ-R-Ye-19(1)-037-3/HZ-R-Ye- |
| | 19(1)-037-4 CDR3 amino acid sequence AWQTSP |
| | RRMMQMAGSIT |
| 4 | R-Ye-19(1)-037 VHH amino acid sequence |
| | QVQLQESGGGLVQAGGSLRLACTVSRGTFSEYSMGWFRQAPGQ |
| | ERDFVTAISGFGHITHYADSVKGRFTISRDNAANTVYLQMNTL |
| | KPEDTAVYYCAAAWQTSPRRMMQMAGSITWGQGTQVTVSS |
| 5 | HZ-R-Ye-19(1)-037-1 VHH amino acid sequence |
| | EVQLLESGGGLVQPGGSLRLSCTVSRGTFSEYSMGWFRQAPGQ |
| | ERDFVTAISGFGHITHYADSVKGRFTISRDNSKNTVYLQMNSL |
| | KPEDTAVYYCAAAWQTSPRRMMQMAGSITWGQGTQVTVSS |
| 6 | HZ-R-Ye-19(1)-037-2 VHH amino acid sequence |
| | EVQLLESGGGLVQPGGSLRLSCAVSRGTFSEYSMSWFRQAPGQ |
| | ERDFVTAISGFGHITHYADSVKGRFTISRDNSKNTVYLQMNSL |
| | RAEDTAVYYCAAAWQTSPRRMMQMAGSITWGQGTQVTVSS |
| 7 | HZ-R-Ye-19(1)-037-3 VHH amino acid sequence |
| | EVQLLESGGGLVQPGGSLRLSCAVSRGTFSEYSMSWFRQAPGK |
| | EREFVTAISGFGHITHYADSVKGRFTISRDNSKNTVYLQMNSL |
| | RAEDTAVYYCAAAWQTSPRRMMQMAGSITWGQGTQVTVSS |
| 8 | HZ-R-Ye-19(1)-037-4 VHH amino acid sequence |
| | EVQLLESGGGLVQPGGSLRLSCAVSRGTFSEYSMSWFRQAPGK |
| | GLEFVTAISGFGHITHYADSVKGRFTISRDNSKNTVYLQMNSL |
| | RAEDTAVYYCAAAWQTSPRRMMQMAGSITWGQGTQVTVSS |
| 9 | R-Ye-19(1)-046/HZ-R-Ye-19(1)-046-1 CDR1 |
| | amino acid sequence INYMG |
| 10 | R-Ye-19(1)-046/HZ-R-Ye-19(1)-046-1/HZ-R-Ye- |
| | 19(1)-046-2/HZ-R-Ye-19(1)-046-3/HZ-R-Ye- |
| | 19(1)-046-4 CDR2 amino acid sequence AITGRG |
| | NTNYADSVKG |
| 11 | R-Ye-19(1)-046/HZ-R-Ye-19(1)-046-1/HZ-R-Ye- |
| | 19(1)-046-3/HZ-R-Ye-19(1)-046-4 CDR3 amino |
| | acid sequence WDYSDYDEGPLREYDY |
| 12 | R-Ye-19(1)-046 VHH amino acid sequence |
| | QVQLQESGGGLVQTGRSLRLSCAASGDIFSINYMGWYRQAPGN |
| | ERELVAAITGRGNTNYADSVKGRFTISRDNAKNTVYLQMNSLK |
| | PEDTAVYYCNAWDYSDYDEGPLREYDYWGQGTQVTVSS |
| 13 | HZ-R-Ye-19(1)-046-1 VHH amino acid sequence |
| | EVQLLESGGGLVQPGRSLRLSCAASGDIFSINYMGWYRQAPGN |
| | ERELVAAITGRGNTNYADSVKGRFTISRDNSKNTVYLQMNSLK |
| | PEDTAVYYCNAWDYSDYDEGPLREYDYWGQGTQVTVSS |
| 14 | HZ-R-Ye-19(1)-046-2/HZ-R-Ye-19(1)-046-3/HZ- |
| | R-Ye-19(1)-046-4 CDR1 amino acid sequence |
| | INYMS |
| 15 | HZ-R-Ye-19(1)-046-2 CDR3 amino acid |
| | sequence WDYSDADEGPLREYDY |
| 16 | HZ-R-Ye-19(1)-046-2 VHH amino acid sequence |
| | EVQLLESGGGLVQPGGSLRLSCAASGDIFSINYMSWYRQAPGN |
| | ERELVAAITGRGNTNYADSVKGRFTISRDNSKNTVYLQMNSLR |
| | AEDTAVYYCNAWDYSDADEGPLREYDYWGQGTQVTVSS |
| 17 | HZ-R-Ye-19(1)-046-3 VHH amino acid sequence |
| | EVQLLESGGGLVQPGGSLRLSCAASGDIFSINYMSWYRQAPGK |
| | ERELVAAITGRGNTNYADSVKGRFTISRDNSKNTVYLQMNSLR |
| | AEDTAVYYCNAWDYSDYDEGPLREYDYWGQGTQVTVSS |
| 18 | HZ-R-Ye-19(1)-046-4 VHH amino acid sequence |
| | EVQLLESGGGLVQPGGSLRLSCAASGDIFSINYMSWYRQAPGK |
| | GLELVAAITGRGNTNYADSVKGRFTISRDNSKNTVYLQMNSLR |
| | AEDTAVYYCNAWDYSDYDEGPLREYDYWGQGTQVTVSS |
| 19 | Bi307/308-H amino acid sequence |
| | EVQLLESGGGLVQPGGSLRLSCAASGDIFSINYMSWYRQAPGN |
| | ERELVAAITGRGNTNYADSVKGRFTISRDNSKNTVYLQMNSLR |
| | AEDTAVYYCNAWDYSDADEGPLREYDYWGQGTQVTVSSASTKG |
| | PSVFPLAPSSKSTSGGTAALGCLVKDYFPEPVTVSWNSGALTS |
| | GVHTFPAVLQSSGLYSLSSVVTVPSSSLGTQTYICNVNHKPSN |
| | TKVDKKVEPKSCDKTHTCPPCPAPEAAGGPSVFLFPPKPKDTL |
| | MISRTPEVTCVVVDVSHEDPEVKFNWYVDGVEVHNAKTKPREE |
| | QYNSTYRVVSVLTVLHQDWLNGKEYKCKVSNKALPAPIEKTIS |
| | KAKGQPREPQVYTLPPSREEMTKNQVSLTCLVKGFYPSDIAVE |
| | WESNGQPENNYKTTPPVLDSDGSFFLYSKLTVDKSRWQQGNVF |
| | SCSVMHEALHNHYTQKSLSLSPG |
| 20 | Bi307/308-L amino acid sequence |
| | EVQLLESGGGLVQPGGSLRLSCAVSRGTFSEYSMSWFRQAPGK |
| | EREFVTAISGFGHITHYADSVKGRFTISRDNSKNTVYLQMNSL |
| | RAEDTAVYYCAAAWQTSPRRMMQMAGSITWGQGTQVTVSSRTV |
| | AAPSVFIFPPSDEQLKSGTASVVCLLNNFYPREAKVQWKVDNA |
| | LQSGNSQESVTEQDSKDSTYSLSSTLTLSKADYEKHKVYACEV |
| | THQGLSSPVTKSFNRGEC |
| 21 | Fc-37-46 amino acid sequence |
| | DKTHTCPPCPAPEAAGGPSVFLFPPKPKDTLMISRTPEVTCVV |
| | VDVSHEDPEVKFNWYVDGVEVHNAKTKPREEQYNSTYRVVSVL |
| | TVLHQDWLNGKEYKCKVSNKALPAPIEKTISKAKGQPREPQVY |
| | TLPPSRDELTKNQVSLTCLVKGFYPSDIAVEWESNGQPENNYK |
| | TTPPVLDSDGSFFLYSKLTVDKSRWQQGNVFSCSVMHEALHNH |
| | YTQKSLSLSPGGGGGAGGGGAGGGGAGGGGAEVQLLESGGGLV |
| | QPGGSLRLSCAVSRGTFSEYSMSWFRQAPGKEREFVTAISGFG |
| | HITHYADSVKGRFTISRDNSKNTVYLQMNSLRAEDTAVYYCAA |
| | AWQTSPRRMMQMAGSITWGQGTQVTVSSGGGGAGGGGAGGGGA |
| | GGGGAGGGGAGGGGAGGGGAEVQLLESGGGLVQPGGSLRLSCA |
| | ASGDIFSINYMSWYRQAPGNERELVAAITGRGNTNYADSVKGR |
| | FTISRDNSKNTVYLQMNSLRAEDTAVYYCNAWDYSDADEGPLR |
| | EYDYWGQGTQVTVSSA |
| 22 | 46-37-Fc amino acid sequence |
| | EVQLLESGGGLVQPGGSLRLSCAASGDIFSINYMSWYRQAPGN |
| | ERELVAAITGRGNTNYADSVKGRFTISRDNSKNTVYLQMNSLR |
| | AEDTAVYYCNAWDYSDADEGPLREYDYWGQGTQVTVSSGGGGA |
| | GGGGAGGGGAGGGGAEVQLLESGGGLVQPGGSLRLSCAVSRGT |
| | FSEYSMSWFRQAPGKEREFVTAISGFGHITHYADSVKGRFTIS |
| | RDNSKNTVYLQMNSLRAEDTAVYYCAAAWQTSPRRMMQMAGSI |
| | TWGQGTQVTVSSDKTHTCPPCPAPEAAGGPSVFLFPPKPKDTL |
| | MISRTPEVTCVVVDVSHEDPEVKFNWYVDGVEVHNAKTKPREE |
| | QYNSTYRVVSVLTVLHQDWLNGKEYKCKVSNKALPAPIEKTIS |
| | KAKGQPREPQVYTLPPSRDELTKNQVSLTCLVKGFYPSDIAVE |
| | WESNGQPENNYKTTPPVLDSDGSFFLYSKLTVDKSRWQQGNVF |
| | SCSVMHEALHNHYTQKSLSLSPG |
| 23 | VH amino acid sequence of anti-PD1 antibody |
| | EVQLVQSGAEVKKPGASVKVSCKASGYTFTEYYIYWVRQAPGQ |
| | GLEWIGGINPSNGGTNFNEKFKPRVTMTVDTSTSTAYMELSSL |
| | RSEDTAVYYCTVRDFRFDKGFKYWGQGTLVTVSS |
| 24 | VL amino acid sequence of anti-PD1 antibody |
| | DIVLTQSPATLSLSPGERATLSCRASKSVSTSGLNYVHWYQRK |
| | PGQAPRLLIYLGSYLDSGVPARFSGSGSGTDFTLTISSLEPED |
| | FAVYYCQQSWELPLTFGGGTKVEIK |
| 25 | Heavy chain amino acid sequence of anti- |
| | PD1xCD39 antibody |
| | EVQLVQSGAEVKKPGASVKVSCKASGYTFTEYYIYWVRQAPGQ |
| | GLEWIGGINPSNGGTNFNEKFKPRVTMTVDTSTSTAYMELSSL |
| | RSEDTAVYYCTVRDFRFDKGFKYWGQGTLVTVSSASTKGPSVF |
| | PLAPSSKSTSGGTAALGCLVKDYFPEPVTVSWNSGALTSGVHT |
| | FPAVLQSSGLYSLSSVVTVPSSSLGTQTYICNVNHKPSNTKVD |
| | KKVEPKSCDKTHTCPPCPAPEAAGAPSVFLFPPKPKDTLMISR |
| | TPEVTCVVVDVSHEDPEVKFNWYVDGVEVHNAKTKPREEQYNS |
| | TYRVVSVLTVLHQDWLNGKEYKCKVSNKALPAPIEKTISKAKG |
| | QPREPQVYTLPPSRDELTKNQVSLTCLVKGFYPSDIAVEWESN |
| | GQPENNYKTTPPVLDSDGSFFLYSKLTVDKSRWQQGNVFSCSV |
| | MHEALHNHYTQKSLSLSPGGGGGAGGGGAGGGGAGGGGAEVQL |
| | LESGGGLVQPGGSLRLSCAVSRGTFSEYSMSWFRQAPGKEREF |
| | VTAISGFGHITHYADSVKGRFTISRDNSKNTVYLQMNSLRAED |
| | TAVYYCAAAWQTSPRRMMQMAGSITWGQGTQVTVSSGGGGAGG |
| | GGAGGGGAGGGGAGGGGAGGGGAGGGGAEVQLLESGGGLVQPG |
| | GSLRLSCAASGDIFSINYMSWYRQAPGNERELVAAITGRGNTN |
| | YADSVKGRFTISRDNSKNTVYLQMNSLRAEDTAVYYCNAWDYS |
| | DADEGPLREYDYWGQGTQVTVSSA |
| 26 | Light chain amino acid sequence of anti- |
| | PD1xCD39 antibody |
| | DIVLTQSPATLSLSPGERATLSCRASKSVSTSGLNYVHWYQRK |
| | PGQAPRLLIYLGSYLDSGVPARFSGSGSGTDFTLTISSLEPED |
| | FAVYYCQQSWELPLTFGGGTKVEIKRTVAAPSVFIFPPSDEQL |
| | KSGTASVVCLLNNFYPREAKVQWKVDNALQSGNSQESVTEQDS |
| | KDSTYSLSSTLTLSKADYEKHKVYACEVTHQGLSSPVTKSFNR |
| | GEC |
| 27 | Amino acid sequence of human IgG1 Fc region |
| | DKTHTCPPCPAPEAAGGPSVFLFPPKPKDTLMISRTPEVTCVV |
| | VDVSHEDPEVKFNWYVDGVEVHNAKTKPREEQYNSTYRVVSVL |
| | TVLHQDWLNGKEYKCKVSNKALPAPIEKTISKAKGQPREPQVY |
| | TLPPSRDELTKNQVSLTCLVKGFYPSDIAVEWESNGQPENNYK |
| | TTPPVLDSDGSFFLYSKLTVDKSRWQQGNVFSCSVMHEALHNH |
| | YTQKSLSLSPG |
| 28 | Amino acid sequence of human CD39 |
| | MEDTKESNVKTFCSKNILAILGFSSIIAVIALLAVGLTQNKAL |
| | PENVKYGIVLDAGSSHTSLYIYKWPAEKENDTGVVHQVEECRV |
| | KGPGISKFVQKVNEIGIYLTDCMERAREVIPRSQHQETPVYLG |
| | ATAGMRLLRMESEELADRVLDVVERSLSNYPFDFQGARIITGQ |
| | EEGAYGWITINYLLGKFSQKTRWFSIVPYETNNQETFGALDLG |
| | GASTQVTFVPQNQTIESPDNALQFRLYGKDYNVYTHSFLCYGK |
| | DQALWQKLAKDIQVASNEILRDPCFHPGYKKVVNVSDLYKTPC |
| | TKRFEMTLPFQQFEIQGIGNYQQCHQSILELFNTSYCPYSQCA |
| | FNGIFLPPLQGDFGAFSAFYFVMKFLNLTSEKVSQEKVTEMMK |
| | KFCAQPWEEIKTSYAGVKEKYLSEYCFSGTYILSLLLQGYHFT |
| | ADSWEHIHFIGKIQGSDAGWTLGYMLNLTNMIPAEQPLSTPLS |
| | HSTYVFLMVLFSLVLFTVAIIGLLIFHKPSYFWKDMV |
| 29 | Amino acid sequence of cynomolgus CD39 |
| | MGREELYLTFSFSSGFRESNVKTFCSKNILAILGFSSIIAVIA |
| | LLAVGLTQNKALPENIKYGIVLDAGSSHTSLYIYKWPAEKEND |
| | TGVVHQVEECRVKGPGISKYVQKVNEIGIYLTDCMERAREVIP |
| | RSQHQETPVYLGATAGMRLLRMESEELADRVLDVVERSLSNYP |
| | FDFQGARIITGQEEGAYGWITINYLLGKFSQKTRWFSIVPYET |
| | NNQETFGALDLGGASTQITFVPQNQTTESPDNALQFRLYGKDY |
| | NVYTHSFLCYGKDQALWQKLAKDIQVASNEILRDPCFHPGYKK |
| | VVNVSDLYKTPCTKRFEMTLPFQQFEIQGIGNYQQCHQSVLEL |
| | FNTSYCPYSQCAFNGIFLPPLQGDFGAFSAFYFVMNFLNLTSE |
| | KVSQEKVTEMMKKFCSQPWEEIKTSYAGVKEKYLSEYCFSGTY |
| | ILSLLLQGYHFTADSWEHIHFIGKIQGSDAGWTLGYMLNLTNM |
| | IPAEQPLSTPLSHSTYVFLMVLFSLVLVIVAIIGLLIFHKPSY |
| | FWKDMV |
| 30 | Human PD-1 amino acid sequence |
| | MQIPQAPWPVVWAVLQLGWRPGWFLDSPDRPWNPPTFSPALLV |
| | VTEGDNATFTCSFSNTSESFVLNWYRMSPSNQTDKLAAFPEDR |
| | SQPGQDCRFRVTQLPNGRDFHMSVVRARRNDSGTYLCGAISLA |
| | PKAQIKESLRAELRVTERRAEVPTAHPSPSPRPAGQFQTLVVG |
| | VVGGLLGSLVLLVWVLAVICSRAARGTIGARRTGQPLKEDPSA |
| | VPVFSVDYGELDFQWREKTPEPPVPCVPEQTEYATIVFPSGMG |
| | TSSPARRGSADGPRSAQPLRPEDGHCSWPL |
| 31 | Cynomolgus PD-1 amino acid sequence |
| | MQIPQAPWPVVWAVLQLGWRPGWFLESPDRPWNPPTFSPALLL |
| | VTEGDNATFTCSFSNASESFVLNWYRMSPSNQTDKLAAFPEDR |
| | SQPGRDCRFRVTQLPNGRDFHMSVVRARRNDSGTYLCGAISLA |
| | PKAQIKESLRAELRVTERRAEVPTAHPSPSPRPAGQFQALVVG |
| | VVGGLLGSLVLLVWVLAVICSRAAQGTIEARRTGQPLKEDPSA |
| | VPVFSVDYGELDFQWREKTPEPPAPCVPEQTEYATIVFPSGLG |
| | TSSPARRGSADGPRSPRPLRPEDGHCSWPL |
| 32 | Light chain constant region amino acid |
| | sequence |
| | RTVAAPSVFIFPPSDEQLKSGTASVVCLLNNFYPREAKVQWKV |
| | DNALQSGNSQESVTEQDSKDSTYSLSSTLTLSKADYEKHKVYA |
| | CEVTHQGLSSPVTKSFNRGEC |
| 33 | Amino acid sequence of heavy chain |
| | constant region |
| | ASTKGPSVFPLAPSSKSTSGGTAALGCLVKDYFPEPVTVSWNS |
| | GALTSGVHTFPAVLQSSGLYSLSSVVTVPSSSLGTQTYICNVN |
| | HKPSNTKVDKKVEPKSCDKTHTCPPCPAPEAAGAPSVFLFPPK |
| | PKDTLMISRTPEVTCVVVDVSHEDPEVKFNWYVDGVEVHNAKT |
| | KPREEQYNSTYRVVSVLTVLHQDWLNGKEYKCKVSNKALPAPI |
| | EKTISKAKGQPREPQVYTLPPSRDELTKNQVSLTCLVKGFYPS |
| | DIAVEWESNGQPENNYKTTPPVLDSDGSFFLYSKLTVDKSRWQ |
| | QGNVFSCSVMHEALHNHYTQKSLSLSPG |
| 34 | 1394 BMK heavy chain amino acid sequence |
| | EVQLQQSGPELVKPGASVKMSCKASGYTFTDYNMHWVKQSHGR |
| | TLEWIGYIVPLNGGSTFNQKFKGRATLTVNTSSRTAYMELRSL |
| | TSEDSAAYYCARGGTRFAYWGQGTLVTVSAASTKGPSVFPLAP |
| | SSKSTSGGTAALGCLVKDYFPEPVTVSWNSGALTSGVHTFPAV |
| | LQSSGLYSLSSVVTVPSSSLGTQTYICNVNHKPSNTKVDKKVE |
| | PKSCDKTHTCPPCPAPEAEGAPSVFLFPPKPKDTLMISRTPEV |
| | TCVVVDVSHEDPEVKFNWYVDGVEVHNAKTKPREEQYNSTYRV |
| | VSVLTVLHQDWLNGKEYKCKVSNKALPSSIEKTISKAKGQPRE |
| | PQVYTLPPSRDELTKNQVSLTCLVKGFYPSDIAVEWESNGQPE |
| | NNYKTTPPVLDSDGSFFLYSKLTVDKSRWQQGNVFSCSVMHEA |
| | LHNHYTQKSLSLSPG |
| 35 | 1394 BMK light chain amino acid sequence |
| | DIVLTQSPASLAVSLGQRATISCRASESVDNFGVSFMYWFQQK |
| | PGQPPNLLIYGASNQGSGVPARFRGSGSGTDFSLNIHPMEADD |
| | TAMYFCQQTKEVPYTFGGGTKLEIKRTVAAPSVFIFPPSDEQL |
| | KSGTASVVCLLNNFYPREAKVQWKVDNALQSGNSQESVTEQDS |
| | KDSTYSLSSTLTLSKADYEKHKVYACEVTHQGLSSPVTKSFNR |
| | GEC |
| 36 | HCDR1 amino acid sequence of anti-PD1 |
| | antibody EYYIY |
| 37 | HCDR2 amino acid sequence of anti-PD1 |
| | antibody GINPSNGGTNFNEKFKP |
| 38 | HCDR3 amino acid sequence of anti-PD1 |
| | antibody RDFRFDKGFKY |
| 39 | LCDR1 amino acid sequence of anti-PD1 |
| | antibody RASKSVSTSGLNYVH |
| 40 | LCDR2 amino acid sequence of anti-PD1 |
| | antibody LGSYLDS |
| 41 | LCDR3 amino acid sequence of anti-PD1 |
| | antibody QQSWELPLT |
| 42 | Heavy chain amino acid sequence of anti- |
| | CD73 antibody |
| | QVQLQESGPGLVKPSQTLSLTCTVSGYSITSGYDWHWIRQHPG |
| | KGLEWMGYISYSGYTDYNPSLKSRITISHDTSKNQFSLKLSSV |
| | TAADTAVYYCTRGDHSYAMDYWGQGTLVTVSSASTKGPSVFPL |
| | APSSKSTSGGTAALGCLVKDYFPEPVTVSWNSGALTSGVHTFP |
| | AVLQSSGLYSLSSVVTVPSSSLGTQTYICNVNHKPSNTKVDKK |
| | VEPKSCDKTHTCPPCPAPEFEGGPSVFLFPPKPKDTLYITREP |
| | EVTCVVVDVSHEDPEVKFNWYVDGVEVHNAKTKPREEQYNSTY |
| | RVVSVLTVLHQDWLNGKEYKCKVSNKALPASIEKTISKAKGQP |
| | REPQVYTLPPSREEMTKNQVSLTCLVKGFYPSDIAVEWESNGQ |
| | PENNYKTTPPVLDSDGSFFLYSKLTVDKSRWQQGNVFSCSVMH |
| | EALHNHYTQKSLSLSPG |
| 43 | Light chain amino acid sequence of anti- |
| | CD73 antibody |
| | DIQMTQSHSSLSASVGDRVTITCKASQAVGTAVAWYQQKPGKS |
| | PKLLIYWASSRHTGVPSRFSGSRSGTDFTLTISSLQPEDFATY |
| | FCQQYSSYPLTFGGGTKVEIKRTVAAPSVFIFPPSDEQLKSGT |
| | ASVVCLLNNFYPREAKVQWKVDNALQSGNSQESVTEQDSKDST |
| | YSLSSTLTLSKADYEKHKVYACEVTHQGLSSPVTKSFNRGEC |

### EXAMPLES

The present invention is described with reference to the following embodiments intended to illustrate the invention (rather than limit the invention).

Unless otherwise specified, the molecular biological experimental method and immunoassays method used herein basically refer to Sambrook, J., et al., Molecular Cloning: A Laboratory Manual, second edition, Cold Spring Harbor Laboratory Press, 1989, and F M. Ausubel et al., Short protocols in molecular biology, 3rd Edition, John Wiley&Sons, Inc., 1995. The restriction endonucleases are used according to the conditions recommended by the manufacturer. It should be appreciated that the following examples are described as illustration and not intended to limit the scope of protection of the invention.

### Example 1: Immunization and Screening of Anti-CD39 Nanobody

Alpaca (Llama) was immune with human CD39 antigen (commercially available from Sinobiological, product No: 16020-H08B). Total RNA was extracted from the peripheral lymphocytes of the alpaca and subjected to reverse transcription to obtain cDNA. The PCR product of cDNA was connected with yeast display vector and then electro-transformed into Saccharomyces cerevisiae cells (commercially available from ATCC, product No: 208289) for constructing anti-CD39 nanobody library.

Human CD39 protein was labelled with biotin labeling kit (commercially available from Thermo, product No: 90407) according to the product instructions. The anti-CD39 nanobody yeast library, after proliferation, was labeled with biotin-labeled CD39 protein, and enriched with magnetic beads for positively labeled yeast cells. After amplification, to the yeast cells enriched with magnetic beads were added 1:200 diluted anti-c-Myc antibody (commercially available from Thermo, product No: MA1-980) and appropriate amount of biotin-labeled CD39 for staining. After washing with PBS, to the yeast cells were added 1:500 diluted Goat-Anti-mouse IgG (H+L) Alexa Fluor Plus 488 (commercially available from Invitrogen, product No: A32723TR) and streptavidin APC Conjugate fluorescent antibody (commercially available from Invitrogen, product No: SA1005), followed by incubate for 15 minutes. The cells were resuspended with PBS and sorted with BD FACSAria II instrument to obtain yeast cells with high binding ability to human CD39.

A liquid of yeast cells with high binding ability to human CD39, resulted from magnetic bead enrichment and flow cytometry sorting, was cultured overnight in the amplification medium at 30° C. and 225 rpm, and subjected to yeast plasmid extraction by yeast plasmid extraction kit (commercially available from Tiangen, product No:: DP112) according to the product instructions. The plasmid was electro-transformed into Top10 receptive cells (commercially available from Tiangen, product No: CB104-02), plated onto ampicillin resistant plate, and cultured overnight at 37° C. Single clone was selected for sequencing to obtain VHH (variable region) gene sequence.

### Example 2 Vector Construction, Protein Expression and Purification of Anti-CD39 Nanobody

The VHH coding sequence of anti-CD39 nanobody obtained from the screening was subjected to homologous recombination with a human IgG1 Fc segment coding sequence (see SEQ ID NO: 27 for amino acid sequence) to construct fusion protein expression sequence. Using ExpiCHO™ Expression System kit (commercially available from Thermo, product No: A2910001), fusion protein expression plasmids from a medium preparation were transformed into Expi-CHO cells (commercially available from Thermo, product No: A2910002) according to the product instructions. After a five days incubation, the supernatant was collected and purified for target protein with sorting procedure using Protein A magnetic beads (commercially available from Genscript Biotech Corporation, product No: L00723). The magnetic beads were suspended in a proper volume (1-4 times the volume of the magnetic beads) of Binding buffer (PBS+0.1% Tween 20, pH 7.4), added to the sample to be purified and incubated at room temperature for 1 hour, with gently shake during the period. The sample was set on a magnetic shelf (commercially available from Beaver Biosciences Inc.). After supernatant was discarded, the magnetic beads were washed with Binding buffer for 3 times, and added with a proper volume (3-5 times the volume of the magnetic beads) of Elution buffer (0.1M sodium citrate, pH 3.2), followed by shaking for 5-10 min at room temperature. After the sample was set back on the magnetic shelf, Elution buffer was collected, transferred to a collection tube containing Neutralization buffer (1M Tris, pH 8.54), and mixed until homogeneity. Thus, the preparation procedure was completed to obtain purified anti-CD39 nanobodies, R-Ye-19 (1)-037 and R-Ye-19 (1)-046, whose CDR and variable region amino acid sequences were shown in Table 1.

### Example 3 Affinity Test of Anti-CD39 Nanobody at Protein Level

ForteBio affinity test was conducted according to the existing methods (Estep, P et al., High throughput solution based measurement of antibody-antigen affinity and affinity binding. MAbs, 2013.5 (2): p. 270-8). In short, the sensor is balanced offline in the analysis buffer for 30 min, and then tested online for 60 s to establish baseline. After that, the AHQ sensor is online loaded with the above-obtained purified antibody, and put into 100 nM human CD39 (SEQ ID NO: 28) for 5 min, and then transferred the sensor to PBS for dissociation for 5 min. A 1:1 binding model is used for the dynamic analysis. The test was performed with control antibody 1394 BMK (The antibody 1394 BMK was derived from humanized monoclonal antibody I-394, disclosed in WO2019068907A1, which was developed by innate pharma and was regarded as most potent CD39 inhibitor antibody in landscape), and the light and heavy chain amino acid sequences of the control antibody 1394 BMK were shown in SEQ ID NO: 34 and 35 respectively. The test results were shown in Table 2.

TABLE 2
Candidate molecular affinity

| Code | KD(M) | Kon(1/Ms) | Koff(1/s) |
|---|---|---|---|
| R-Ye-19(1)-037 | 9.68E−09 | 1.25E+05 | 1.00E−04 |
| R-Ye-19(1)-046 | 1.24E−09 | 4.21E+05 | 5.29E−04 |
| I394 BMK | 8.55E−10 | 2.85E+05 | 2.00E−04 |

### Example 4 Affinity Test of Anti-CD39 Nanobody at Cell Level

CHO cells overexpressing human CD39 (CHO-hCD39 cells) were produced by transfection of pCHO1.0 vector (commercially available from Invitrogen, product No: HG-VPI0983) with CD39 cDNA. The expanded CHO-CD39 cells were adjusted to a cell density of 2×10⁶ cells/ml, added to 96-well flow plate at 100 μL/well, and centrifuged for future use. The purified anti-CD39 antibody prepared according to Example 2 was diluted with PBS, by triple dilution starting from 400 nM for a total of 12 concentration values. The diluted samples were respectively added at 100 μL/well to the above-obtained 96-well flow plate containing cells. After incubation at 4° C. for 30 minutes, the plate was washed with PBS twice, then added at 100 μL/well with Goat F(ab′)2 Anti-Human IgG-Fc (PE) (commercially available from Abcam, ab98596) diluted 100 times with PBS. After incubation at 4° C. for 30 minutes, the plate was washed with PBS twice, then added at 100 μL/well with PBS and resuspend the cells, and tested on CytoFlex (Bechman) flow cytometry, followed by calculation of corresponding MFI values.

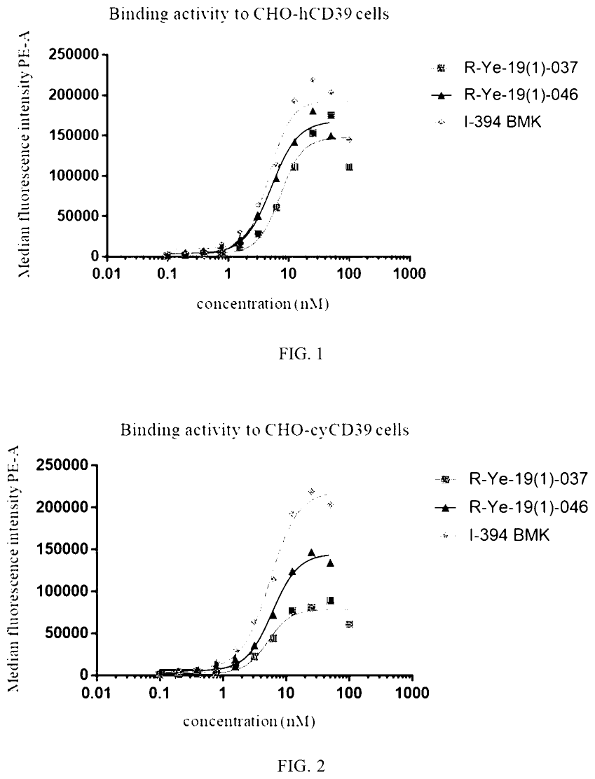

As determined in the above described method, the test results were shown in FIG. 1 and Table 3, indicating that all purified anti-CD39 antibody samples of the invention have binding activity to CHO-hCD39 cells.

TABLE 3
Affinity test results of anti-CD39 nanobody
to CHO-hCD39 at cell level, EC50

| Code | EC50(nM) |
|---|---|
| R-Ye-19(1)-037 | 7.225 |
| R-Ye-19(1)-046 | 5.143 |
| I394 BMK | 4.759 |

In order to identify the affinity of antibodies to cynomolgus CD39 (cyCD39) at the cell level, CHO cell line overexpressing cynomolgus CD39 (CHO-cyCD39 cell) was constructed according to the method described above, and subjected to the affinity test of purified antibodies to CHO-cyCD39 at the cell level. The test results were shown in FIG. 2 and Table 4.

TABLE 4
Affinity test results of anti-CD39 nanobody
to CHO-cyCD39 at cell level, EC50

| Code | EC50(nM) |
|---|---|
| R-Ye-19(1)-037 | 5.131 |
| R-Ye-19(1)-046 | 6.000 |
| I394 BMK | 5.534 |

### Example 5 Determination of Epitope Bin of Anti-CD39 Nanobody

An anti-human IgG Fc sensor was firstly loaded with 100 nM anti-CD39 antibody, baselined for 30 s, quenched with Rituximab for 10 minutes, then loaded with 100 nM human CD39 recombinant protein for 30 s, baselined for 120 s, and then add with another antibody to be tested. If there is a further response, they are antibodies of different Bins, and if there is no response, they are antibodies of the same Bin. Such cycle is repeated until all antibody are classified by Bin.

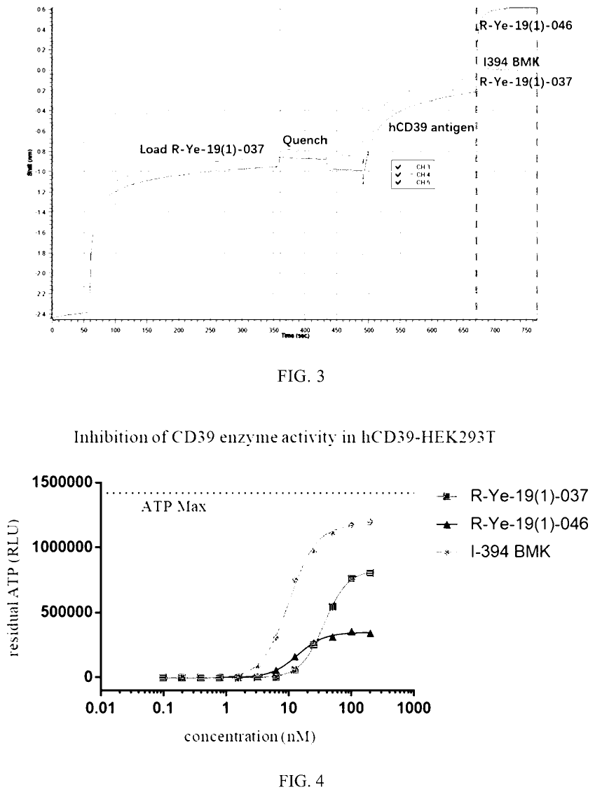

The results were shown in FIG. 3, indicating that R-Ye-19 (1)-037 and 1394 BMK are of the same Bin, while R-Ye-19 (1)-046 is of a different Bin.

### Example 6 Test of Anti-CD39 Nanobodies for Blocking CD39 Enzyme Activity

To wells of a 96-well plate was added huCD39 overexpression cells at 100,000 cells/well. The plate was centrifuged to remove the supernatant, added with the purified anti-CD39 antibody prepared according to Example 2 (for dose dependence: starting from 200 nM, 3 times dilution, 12 concentration values) at 100 μL/well, and incubated at 4° C. for 30 min. The plate was added with 20 μM ATP at 100 μL/well, incubated at 37° C. for 30 min, and centrifuged. The supernatant was added at 50 μL/well to a white 96-well plate, then the white 96-well plate was added with CellTiter-Glo (CTG) at 50 μL/well, and then read under Spectra i3x for chemiluminescence.

The results were shown in FIG. 4 and Table 5, indicating that both of the two candidate anti-CD39 nanobodies are capable of inhibiting the CD39 enzyme activity on the surface of CD39 overexpression cells.

TABLE 5
Activities of anti-CD39 nanobodies for blocking CD39
enzyme activity in overexpression cells, EC50

| Code | EC50(nM) |
|---|---|
| R-Ye-19(1)-037 | 36.48 |
| R-Ye-19(1)-046 | 13.75 |
| I394 BMK | 10.07 |

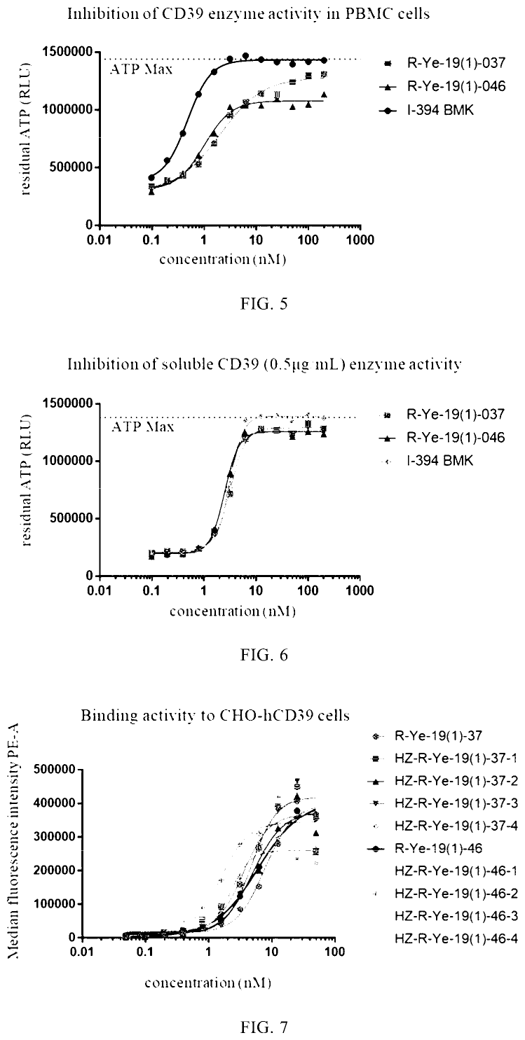

By referring to the above experimental methods, activities for inhibiting human CD39 enzyme activity in PBMC system were tested. The results were shown in FIG. 5 and Table 6, indicating that both of the two candidate anti-CD39 nanobodies are capable of inhibiting the CD39 enzyme activity in PBMC.

TABLE 6
Activities of anti-CD39 nanobodies for blocking
CD39 enzyme activity in PBMC system, EC50

| Code | EC50(nM) |
|---|---|
| R-Ye-19(1)-037 | 36.48 |
| R-Ye-19(1)-046 | 13.75 |
| I394 BMK | 10.07 |

By referring to the above experimental methods, activities for inhibiting CD39 enzyme activity of soluble human CD39 were tested. The results were shown in FIG. 6 and Table 7, indicating that both of the two candidate anti-CD39 nanobodies are capable of inhibiting the CD39 enzyme activity of soluble CD39.

TABLE 7
Activities of anti-CD39 nanobodies for blocking
CD39 enzyme activity of soluble CD39, EC50

| Code | EC50(nM) |
|---|---|
| R-Ye-19(1)-037 | 3.15 |
| R-Ye-19(1)-046 | 2.485 |
| I394 BMK | 3.094 |

### Example 7 Affinity Test of Humanized Anti-CD39 Nanobodies

The sequences of the two antibodies obtained from the screening, i.e., R-Ye-19 (1)-037 and R-Ye-19 (1)-046, was humanized. The humanized sequences are subjected to the humanized antibody vector construction, expression and purification according to the method described in Example 2. Finally, four humanized antibodies were obtained from R-Ye-19 (1)-037, respectively coded by HZ—R-Ye-19 (1)-037-1, HZ—R—Ye-19 (1)-037-2, HZ—R—Ye-19 (1)-037-3 and HZ—R-Ye-19 (1)-037-4; and four humanized modified antibodies were obtained from R-Ye-19 (1)-046, respectively coded by HZ—R-Ye-19 (1)-046-1, HZ—R—Ye-19 (1)-046-2, HZ—R—Ye-19 (1)-046-3 and HZ—R-Ye-19 (1)-046-4. The CDR and variable region amino acid sequences of each of these humanized antibodies were shown in Table 1. The purified humanized antibodies were subjected to the affinity test at protein level according to the method described in Example 3. The results were shown in Table 8.

TABLE 8
Affinity test results of humanized
anti-CD39 nanobody at protein level

| Code | KD(M) | Kon(1/Ms) | Koff(1/s) |
|---|---|---|---|
| R-Ye-19(1)-037 | 3.50E−09 | 3.11E+05 | 1.08E−03 |
| HZ-R-Ye-19(1)-037-1 | 4.10E−09 | 2.62E+05 | 1.09E−03 |
| HZ-R-Ye-19(1)-037-2 | 7.60E−09 | 2.61E+05 | 1.98E−03 |
| HZ-R-Ye-19(1)-037-3 | 7.70E−09 | 2.53E+05 | 1.95E−03 |
| HZ-R-Ye-19(1)-037-4 | 8.60E−09 | 2.34E+05 | 2.02E−03 |
| R-Ye-19(1)-046 | 6.30E−10 | 2.89E+05 | 1.81E−04 |
| HZ-R-Ye-19(1)-046-1 | 4.30E−10 | 3.55E+05 | 1.53E−04 |
| HZ-R-Ye-19(1)-046-2 | 3.40E−09 | 2.47E+05 | 8.29E−04 |
| HZ-R-Ye-19(1)-046-3 | 2.50E−09 | 3.34E+05 | 8.42E−04 |
| HZ-R-Ye-19(1)-046-4 | 3.40E−09 | 2.78E+05 | 9.56E−04 |
| I394 BMK | 1.50E−09 | 2.45E+05 | 3.71E−04 |

### Example 8 Affinity Test of Humanized Anti-CD39 Nanobodies to CHO-hCD39 Cells and their Species Cross-Reactivity

The purified humanized antibodies were subjected to the affinity test to CHO-hCD39 at cell level according to the method described in Example 4. The results were shown in FIG. 7 and Table 9, indicating that the humanized antibodies possess equivalent cell-binding activity to the non-humanized antibodies.

TABLE 9
Affinity of humanized nanobodies
to human CHO-hCD39 cells, EC50

| Code | EC50(nM) |
|---|---|
| R-Ye-19(1)-037 | 7.7 |
| HZ-R-Ye-19(1)-037-1 | 3.50 |
| HZ-R-Ye-19(1)-037-2 | 4.976 |
| HZ-R-Ye-19(1)-037-3 | 4.751 |
| HZ-R-Ye-19(1)-037-4 | 2.196 |
| R-Ye-19(1)-046 | 5.963 |
| HZ-R-Ye-19(1)-046-1 | 2.735 |
| HZ-R-Ye-19(1)-046-2 | 1.552 |
| HZ-R-Ye-19(1)-046-3 | 3.771 |
| HZ-R-Ye-19(1)-046-4 | 2.51 |

In order to identify the affinity of the humanized antibodies to cynomolgus CD39 (cyCD39) at the cell level, a cell line (CHO-cyCD39) was constructed according to the method described in Example 4 and subjected to the affinity test of the purified humanized antibodies to CHO-cyCD39 at the cell level. The results were shown in FIG. 8 and Table 10.

TABLE 10
Affinity of humanized nanobodies to
cynomolgus CHO-cyCD39 cells, EC50

| Code | EC50(nM) |
|---|---|
| R-Ye-19(1)-037 | 4.943 |
| HZ-R-Ye-19(1)-037-1 | 2.526 |
| HZ-R-Ye-19(1)-037-2 | 2.528 |
| HZ-R-Ye-19(1)-037-3 | 3.381 |
| HZ-R-Ye-19(1)-037-4 | 4.821 |
| R-Ye-19(1)-046 | 3.03 |
| HZ-R-Ye-19(1)-046-1 | 3.267 |
| HZ-R-Ye-19(1)-046-2 | 3.978 |
| HZ-R-Ye-19(1)-046-3 | 2.228 |
| HZ-R-Ye-19(1)-046-4 | 2.412 |

### Example 9 Test of Humanized Anti-CD39 Nanobodies for Blocking CD39 Enzyme Activity

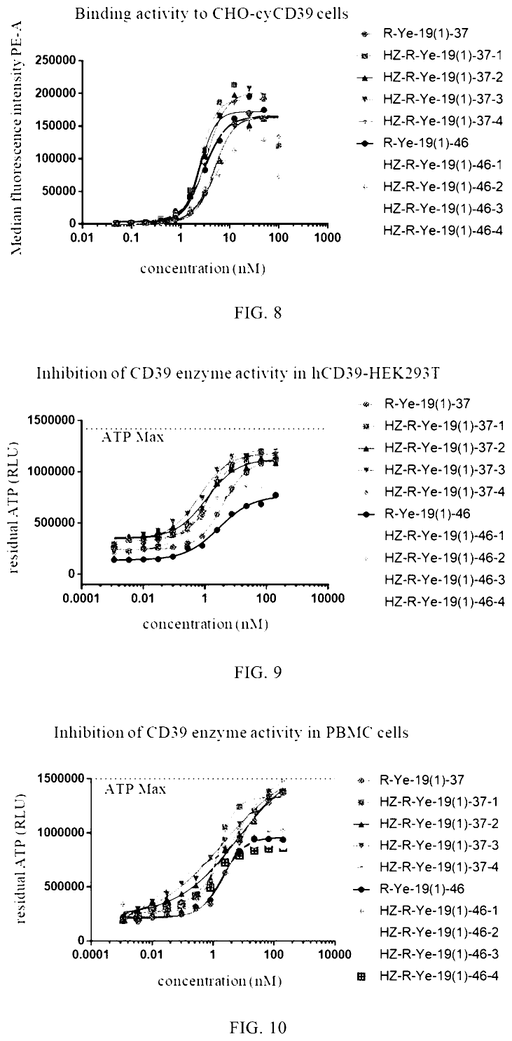

By referring to the method described in Example 6, humanized anti-CD39 nanobodies were tested for the activities of blocking and inhibiting the CD39 enzyme activity on the surface of human CD39 overexpression cells. The results were shown in FIG. 9 and Table 11, indicating that the blocking activities of humanized nanobodies are slightly better than those of parental antibody.

TABLE 11
Activities of humanized nanobodies for blocking
CD39 enzyme activity of overexpression cells

| Code | EC50(nM) |
|---|---|
| R-Ye-19(1)-037 | 3.787 |
| HZ-R-Ye-19(1)-037-1 | 1.407 |
| HZ-R-Ye-19(1)-037-2 | 1.137 |
| HZ-R-Ye-19(1)-037-3 | 0.6592 |
| HZ-R-Ye-19(1)-037-4 | 1.506 |
| R-Ye-19(1)-046 | 2.867 |
| HZ-R-Ye-19(1)-046-1 | 0.9684 |
| HZ-R-Ye-19(1)-046-2 | 0.5487 |
| HZ-R-Ye-19(1)-046-3 | 0.6062 |
| HZ-R-Ye-19(1)-046-4 | 0.9819 |

Similarly, by referring to the method described in Example 6, humanized anti-CD39 nanobodies were further tested for the activities of blocking and inhibiting the human CD39 enzyme activity in PBMC system. The results were shown in FIG. 10 and Table 12, indicating that some humanized nanobodies are slightly better than parental antibodies.

TABLE 12
Activities of humanized nanobodies for blocking
CD39 enzyme activity in PBMC system, EC50

| Code | EC50(nM) |
|---|---|
| R-Ye-19(1)-037 | 4.95 |
| HZ-R-Ye-19(1)-037-1 | 1.412 |
| HZ-R-Ye-19(1)-037-2 | 6.763 |
| HZ-R-Ye-19(1)-037-3 | 2.15 |
| HZ-R-Ye-19(1)-037-4 | 3.974 |
| R-Ye-19(1)-046 | 2.041 |
| HZ-R-Ye-19(1)-046-1 | 1.568 |
| HZ-R-Ye-19(1)-046-2 | 0.02879 |
| HZ-R-Ye-19(1)-046-3 | ~1.05e−005 |
| HZ-R-Ye-19(1)-046-4 | 0.8972 |

Similarly, by referring to the method described in Example 6, humanized anti-CD39 nanobodies were further tested for the activities of blocking enzyme activity of soluble human CD39. The results were shown in FIG. 11 and Table 13, indicating that the humanized nanobody is slightly better than the parental antibody.

TABLE 13
Activities of humanized nanobodies for blocking
enzyme activity of soluble human CD39, EC50

| Code | EC50(nM) |
|---|---|
| R-Ye-19(1)-037 | 18.55 |
| HZ-R-Ye-19(1)-037-1 | 17.34 |
| HZ-R-Ye-19(1)-037-2 | 19.5 |
| HZ-R-Ye-19(1)-037-3 | 18.08 |
| HZ-R-Ye-19(1)-037-4 | 19.43 |
| R-Ye-19(1)-046 | 16.61 |
| HZ-R-Ye-19(1)-046-1 | 17.19 |
| HZ-R-Ye-19(1)-046-2 | 19.67 |
| HZ-R-Ye-19(1)-046-3 | 20.56 |
| HZ-R-Ye-19(1)-046-4 | 22.02 |

### Example 10 Test of the Combination of Two Humanized Anti-CD39 Nanobodies Having Different Epitopes for Blocking CD39 Enzyme Activity

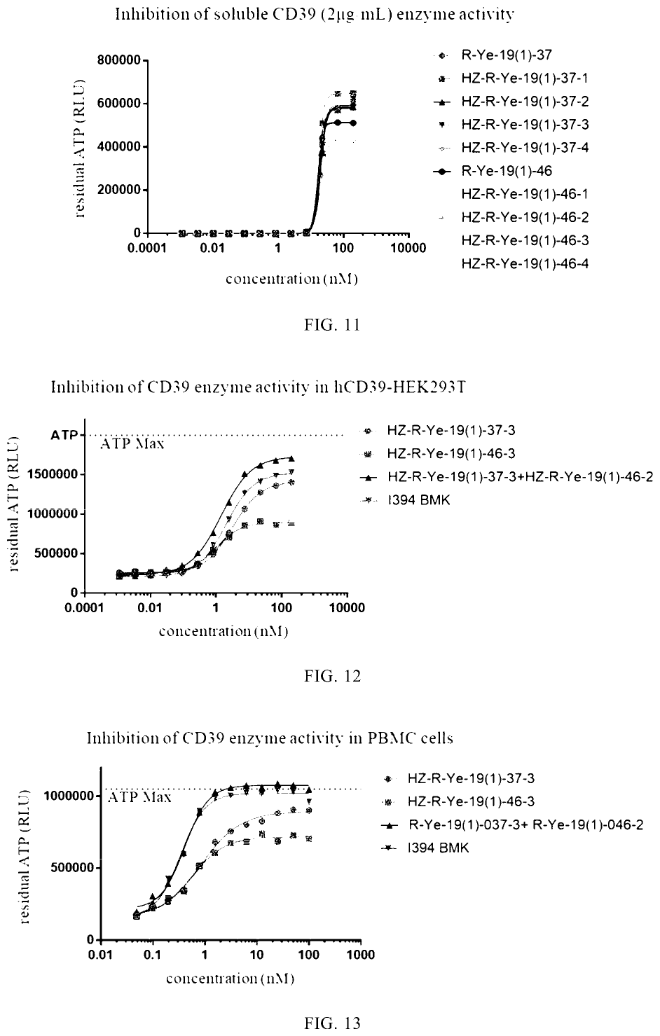

By referring to the method described in Example 6, the combination of two humanized anti-CD39 nanobodies having different epitopes (i.e., HZ—R—Ye-19 (1)-037-3 and HZ—R-Ye-19 (1)-046-2, the molar concentration ratio of HZ—R-Ye-19 (1)-037-3 to HZ—R-Ye-19 (1)-046-2 is 1:1) was test for activity for blocking and inhibiting human CD39 enzyme activity on the surface of overexpression cells. The results were shown in FIG. 12 and Table 14, indicating that there was a strong synergistic effect between the two antibodies having different epitopes, i.e., HZ—R—Ye-19 (1)-037-3 and HZ—R-Ye-19 (1)-046-2, and the combination resulted in better results than the control antibody.

TABLE 14
Activities of humanized nanobodies for blocking
CD39 enzyme activity of overexpression cells

| Code | EC50(nM) |
|---|---|
| HZ-R-Ye-19(1)-037-3 | 3.15 |
| HZ-R-Ye-19(1)-046-2 | 1.01 |
| HZ-R-Ye-19(1)-037-3 + HZ-R-Ye-19(1)-046-2 | 1.426 |
| I394 BMK | 1.986 |

Similarly, by referring to the method described in Example 6, the combination of two humanized anti-CD39 nanobodies having different epitopes (i.e., HZ—R—Ye-19 (1)-037-3 and HZ—R-Ye-19 (1)-046-2, the molar concentration ratio of HZ—R-Ye-19 (1)-037-3 to HZ—R-Ye-19 (1)-046-2 is 1:1) was test for activity for blocking and inhibiting the human CD39 enzyme activity in PBMC system. The results were shown in FIG. 13 and Table 15, indicating that there was a strong synergistic effect between the two antibodies having different epitopes, i.e., HZ—R—Ye-19 (1)-037-3 and HZ—R-Ye-19 (1)-046-2.

TABLE 15
Activities of humanized nanobodies for blocking
CD39 enzyme activity in PBMC system, EC50

| Code | EC50(nM) |
|---|---|
| HZ-R-Ye-19(1)-037-3 | 0.8184 |
| HZ-R-Ye-19(1)-046-2 | 0.5653 |
| HZ-R-Ye-19(1)-037-3 + HZ-R-Ye-19(1)-046-2 | 0.4083 |
| I394 BMK | 0.3378 |

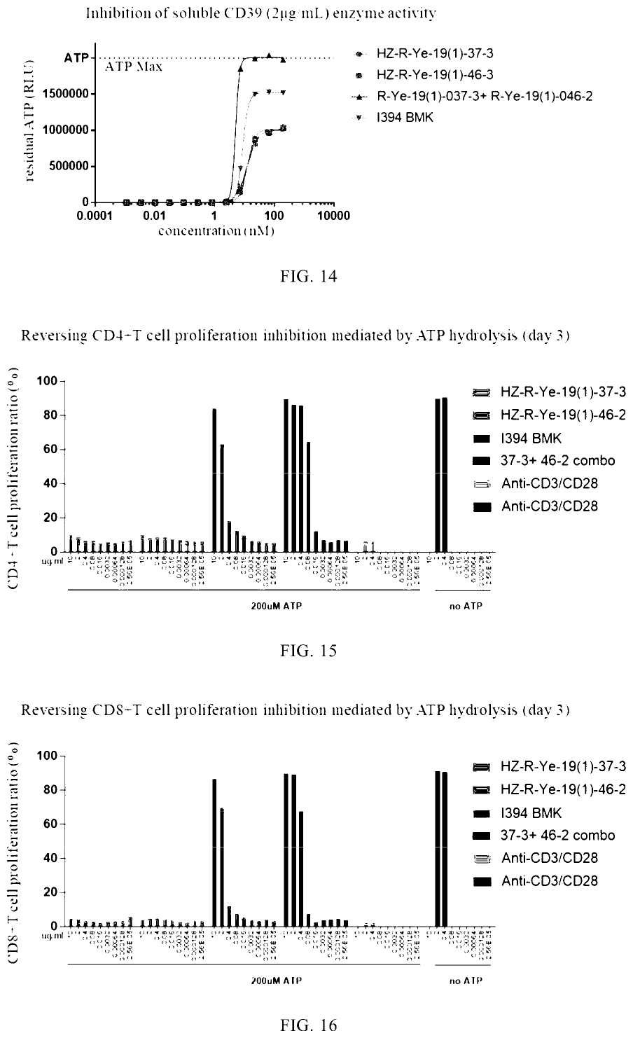

Similarly, by referring to the method described in Example 6, the combination of two humanized anti-CD39 nanobodies having different epitopes (i.e., HZ—R—Ye-19 (1)-037-3 and HZ—R-Ye-19 (1)-046-2, the molar concentration ratio of HZ—R-Ye-19 (1)-037-3 to HZ—R-Ye-19 (1)-046-2 is 1:1) was test for activity for blocking enzyme activity of soluble human CD39. The results were shown in FIG. 14 and Table 16, indicating that there was a strong synergistic effect between the two antibodies having different epitopes, i.e., HZ—R—Ye-19 (1)-037-3 and HZ—R-Ye-19 (1)-046-2, and the combination resulted in better results than the control antibody.

TABLE 16
Activities of humanized nanobodies for blocking
enzyme activity of soluble human CD39, EC50

| Code | EC50(nM) |
|---|---|
| HZ-R-Ye-19(1)-037-3 | 12.58 |
| HZ-R-Ye-19(1)-046-2 | 12.42 |
| HZ-R-Ye-19(1)-037-3 + HZ-R-Ye-19(1)-046-2 | 5.048 |
| I394 BMK | 8.888 |

### Example 11 Test of the Combination of Two Humanized Anti-CD39 Nanobodies Having Different Epitopes for Reversing T Cell Proliferation Inhibition

The experiment was conducted as follows: cryopreserved PBMC cells (commercially available from Sailybio, product No: XFB-HP100B) were thawed and resuspend in X-VIVO15 (commercially available from Lonza, product No: 04-418Q), added with small amount of DNase, and transferred into a T75 square flask. The flask was put into an incubator at 37° C. and incubated for 2 hours to adhere to the flask wall. The suspended cells were pipetted from the above culture bottle, centrifuged at 400×g for 5 min, added 1000-times PBS diluted CTV (commercially available from Invitrogen product No: C34557) per 10⁸ cells, and incubated at 37° C. for 10 min. After washed with PBS twice the cell density was adjusted to 6.0×10⁶ with X-VIVO15 culture medium. At the same time, a 96-well flat bottom plate was coated with PBS diluted 1 μg/mL Anti-human CD3 OKT-3 (commercially available from Biogene, product No: 317348) at 100 μL/well, incubated in an incubator at 37° C. for 2 hours, and rinsed with PBS at 100 μL/well twice, with supernatant discarded. The resulting CTV labeled cells were added at 50 μL/well, together with gradient diluted anti-CD39 antibody sample at 50 μL/well, to the 96-well flat bottom plate, and incubated in an incubator at 37° C. for 1 h.

ATP was diluted with X-VIVO15 medium to a working concentration of 1500 UM and added with purified anti-human CD28 (commercially available from BioLegend, product No: 302902) to a working concentration of 3 μg/mL. The resulting liquid mixture was added at 50 μL/well to the 96-well flat bottom plate coated with 1 μg/mL anti-Human CD3, and incubated in an incubator at 37° C. for 3-5 days. Wells without ATP added was used as positive control well. Flow cytometry was used for determining the proliferation ratio of CTV labeled T cells.

Results were shown in FIG. 15 and FIG. 16, indicating that there was a strong synergistic effect between the two antibodies having different epitopes, i.e., HZ—R—Ye-19 (1)-037-3 and HZ—R-Ye-19 (1)-046-2, and the combination maximally reversed T cell proliferation inhibition and resulted in better results than the control antibody.

### Example 12 Construction and Affinity Test of Bi-Epitopic Anti-CD39 Antibodies

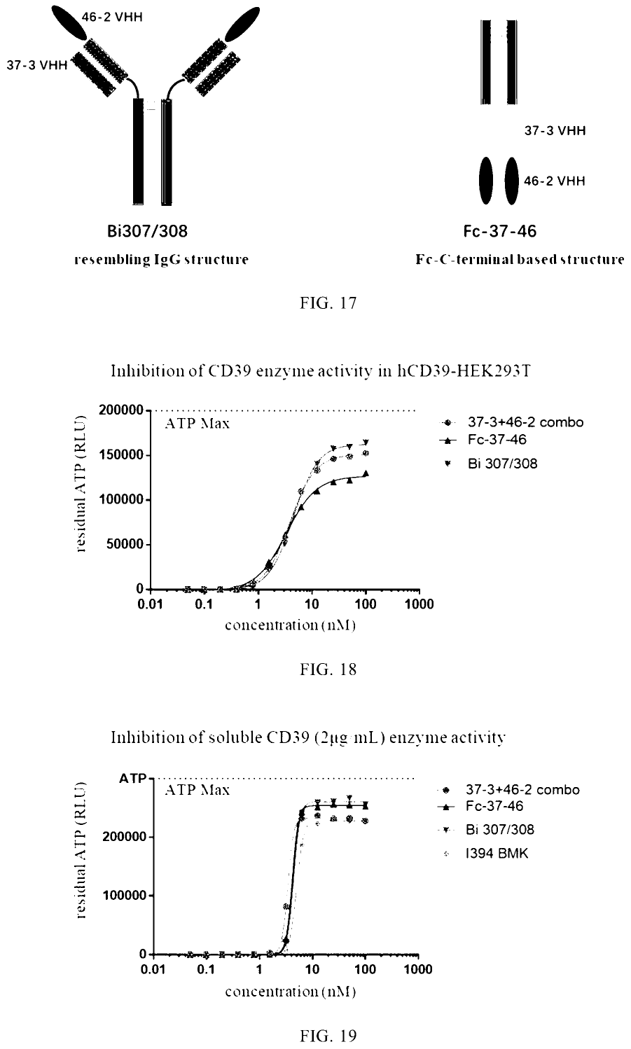

Two kinds of bi-epitopic antibodies were designed, with structures shown in FIG. 17. One had a structure resembling IgG structure (named Bi307/308), and the other had an Fc-C-terminal structure (named Fc-37-46). Bi307/308 is composed of two peptide chains I-A and two peptide chains I-B, wherein the peptide chain I-A comprises, in order of from N terminal to C terminal: HZ—R-Ye-19 (1)-037-3 VHH (SEQ ID NO: 7)-light chain constant region CL (SEQ ID NO: 32), and the peptide chain I-B comprises, in order of from N terminal to C terminal: HZ—R-Ye-19 (1)-046-2 VHH (SEQ ID NO: 16)-heavy chain constant region CH (SEQ ID NO: 33). Fc-37-46 is composed of two peptide chains II, wherein the peptide chain II comprises, in order of from N terminal to C terminal: monomeric Fc domain (SEQ ID NO: 27)-HZ—R-Ye-19 (1)-037-3 VHH (SEQ ID NO: 7)-HZ—R-Ye-19 (1)-046-2 VHH (SEQ ID NO: 16).

The affinity test at protein level was conducted according to the method described in Example 3, and the results were shown in Table 17.

TABLE 17
Affinity of bi-epitopic antibody to human CD39

| Code | KD(M) | Kon(1/Ms) | Koff(1/s) |
|---|---|---|---|
| R-Ye-19(1)-037 | 6.81E−09 | 2.89E+05 | 1.97E−03 |
| R-Ye-19(1)-046 | 2.72E−09 | 3.46E+05 | 9.40E−04 |
| Bi307/308 | 3.51E−10 | 3.03E+05 | 1.06E−04 |
| Fc-37-46 | 3.74E−10 | 3.05E+05 | 1.14E−04 |

### Example 13 Test of Bi-Epitopic Anti-CD39 Antibodies Blocking CD39 Enzyme Activity

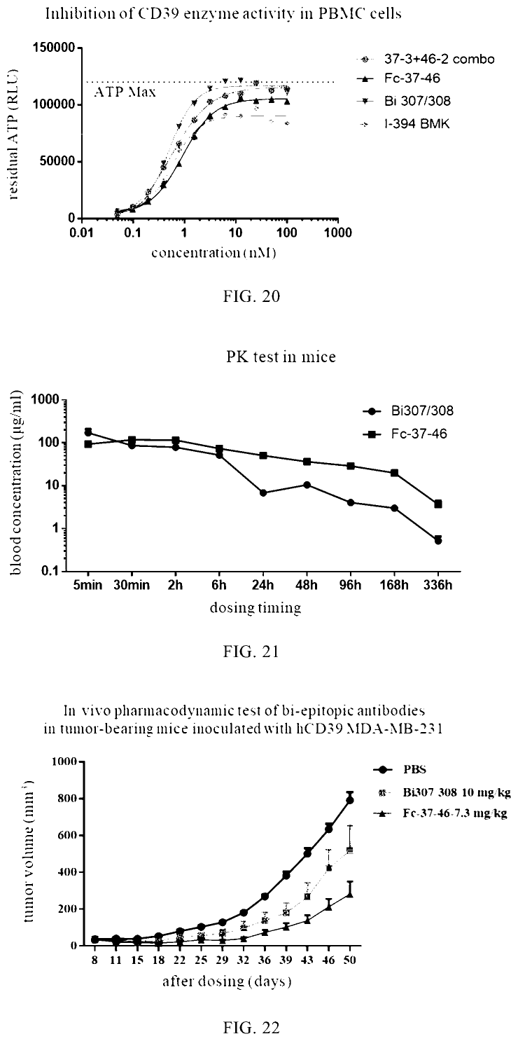

By referring to the method described in Example 6, bi-epitopic anti-CD39 antibodies were tested for blocking human CD39 enzyme activity in different systems. The results are respectively shown in FIGS. 18-20, indicating that, of the bi-epitopic antibodies, Bi307/308 resembling IgG structure resulted in an activity comparable to or slightly superior to the combination group, Fc-37-46 with Fc-C-terminal structure resulted in an activity comparable to the combination group, and both of them are superior to the positive control antibody 1394 BMK.

### Example 14 PK Test of Bi-Epitopic Anti-CD39 Antibodies in Mice

Balb/c mice, half male and half female, were maintained at 12/12 light/dark cycle, temperature 24±2° C., humidity 40-70%, with food and water ad libitum. On the day of the experiment, the Balb/c mice were single injected with specific antibody molecule through tail vein at a dose of 10 mg/kg.

Blood samples were taken from mice orbit at the following timing after dosing: 5 minutes, 0.5 hours, 2 hours, 6 hours, 24 hours, 48 hours, 96 hours, 168 hours, 336 hours, and 504 hours. The whole blood samples were left at 2-8° C. for 30 minutes, followed by centrifugation at 12000 rpm for 5 minutes for serum collection. The obtained serum was then centrifuged at 12000 rpm for 5 minutes at 2-8° C., stored at −80° C. for further determination of blood concentration of anti-CD39 antibody in the serum by ELISA. The results were shown in FIG. 21, indicating that the half-life of Bi307/308 and Fc-37-46 in mice was respectively about 69 hours and 141 hours, and the blood concentration of Bi307/308 in mice dropped rapidly.

### Example 15 Pharmacodynamic Test of Bi-Epitopic Anti-CD39 Antibodies for Anti-Tumor Efficacy in Tumor-Bearing Mice Inoculated with MDA-MB-231 Cell Overexpressing hCD39

In this experiment, anti-CD39 antibodies were tested for anti-tumor efficacy using MDA-MB-231 cells overexpressing hCD39 in B2M KO NDG mice. First, the mice were subcutaneous inoculated with MDA-MB-231 cells overexpressing hCD39 to establish the tumor-bearing mice model. When tumors grew to about 50-60 mm³, the mice were injected intraperitoneally with different doses of different antibodies. The mice in each group were monitored for tumor volume and body weight changes at an interval of 3-4 days for 6-7 weeks. The dosing amounts and administration modes were shown in Table 18. The results were shown in FIG. 22, indicating that, Fc-37-46 resulted in better in-vivo anti-tumor effect in this tumor model over Bi307/308.

TABLE 18
Experimental protocols for tumor inhibition activities

| Group | Dosage | | Dosing Times |
|---|---|---|---|
| Negative control (PBS) | N/A | | Q2W*6 |
| Bi307/308 | 10 | mg/kg | Q2W*6 |
| Fc-37-46 | 7.3 | mg/kg | Q2W*6 |

### Example 16 Pharmacodynamic Test of Bi-Epitopic Anti-CD39 Antibodies for Anti-Tumor Efficacy in Tumor-Bearing Mice Inoculated with Molp-8 Tumor Cell

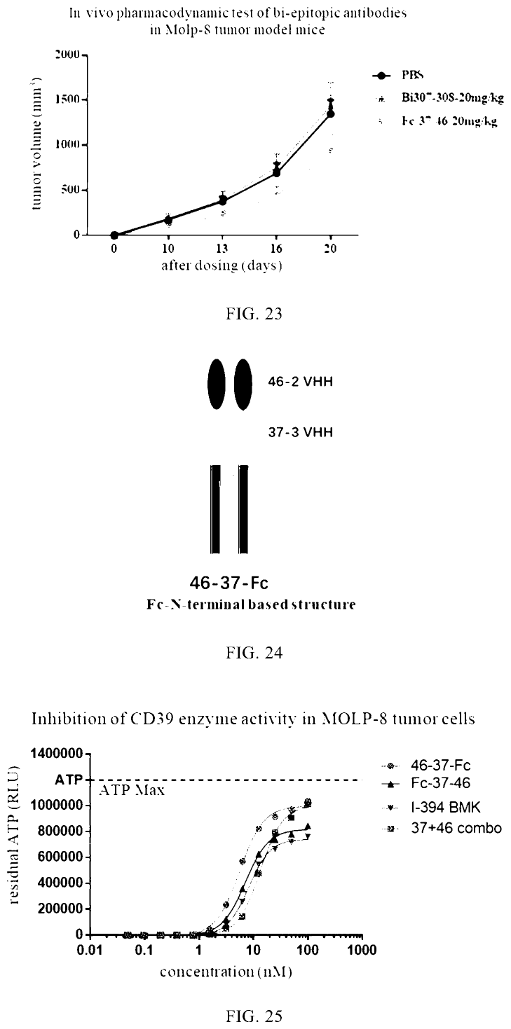

In this experiment, anti-CD39 antibodies were tested for anti-tumor efficacy using Molp-8 cells in CB-17 SCID mice. First, the mice were subcutaneous inoculated to establish Molp-8 tumor cell tumor-bearing mice model, and meanwhile treated with intraperitoneal injection of different doses of different antibodies. The mice in each group were monitored for tumor volume and body weight changes at an interval of 3-4 days for 6-7 weeks. The dosing amounts and administration modes were shown in Table 19. The results were shown in FIG. 23, indicating that, Fc-37-46 resulted in better in-vivo anti-tumor effect in this tumor model over Bi307/308, and Bi307/308 resulted in little effect.

The pharmacodynamic results from the two tumor animal models indicated that Bi307/308 had weak anti-tumor effect. Based on its PK data in mice, it was preliminarily judged that Bi307/308 had low blood concentration in mice, leading to an unsustainable pharmacodynamic effect.

TABLE 19
Experimental protocols for tumor inhibition activities

| Group | Dosage | Dosing Times |
|---|---|---|
| Negative control (PBS) | N/A | Weekly*6 |
| Bi307/308 | 20 mg/kg | Weekly*6 |
| Fc-37-46 | 15 mg/kg | Weekly*6 |

### Example 17 Construction and Affinity Test of Bi-Epitopic Anti-CD39 Antibody with N-Terminal Based Structure

As indicated by the results from previous experiments, Bi307/308 resembling IgG structure had non-obvious anti-tumor effect due to its poor PK properties in mice. Therefore, we redesigned a bi-epitopic antibody with N-terminal based structure (named 46-37-Fc), having a structure shown in FIG. 24, composed of two peptide chains II, wherein the peptide chain II comprises, in order of from N terminal to C terminal: HZ—R-Ye-19 (1)-046-2 VHH (SEQ ID NO: 16)-HZ—R-Ye-19 (1)-037-3 VHH (SEQ ID NO: 7)-monomeric Fc domain (SEQ ID NO: 27).

The bi-epitopic antibody i.e., 46-37-Fc, with N-terminal based structure, was subjected to the affinity test at protein level according to the method described in Example 3, and the results were shown in Table 20.

TABLE 20
Affinity of bi-epitopic antibody to human CD39

| Code | KD(M) | Kon(1/Ms) | Koff(1/s) |
|---|---|---|---|
| 46-37-Fc | 1.60E−09 | 1.30E+05 | 2.11E−04 |

### Example 18 Test of Bi-Epitopic Anti-CD39 Antibody with N-Terminal Based Structure for Blocking CD39 Enzyme Activity

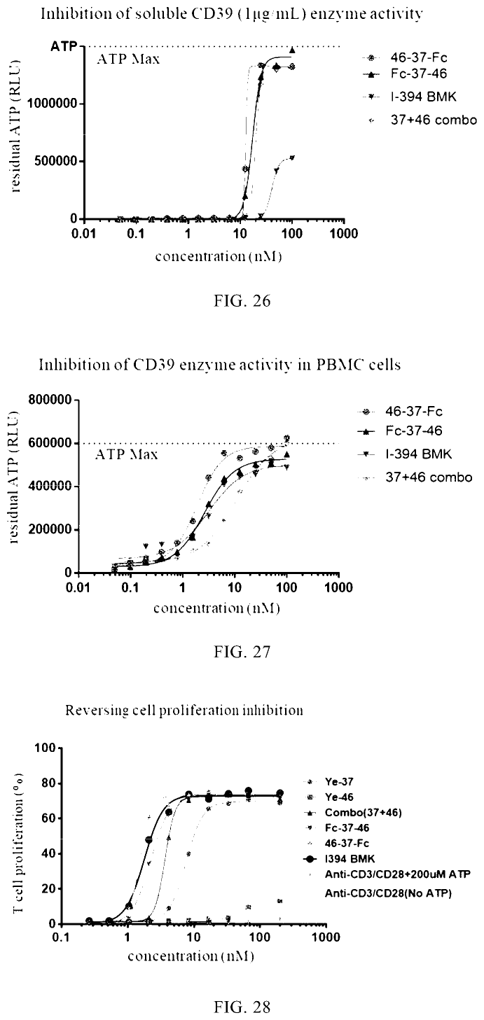

By referring to the method described in Example 6, bi-epitopic anti-CD39 antibodies were tested for blocking human CD39 enzyme activity in different systems. The results are respectively shown in FIGS. 25-27, indicating that, of the bi-epitopic antibodies, the activity of bi-epitopic antibody 46-37-Fc with N-terminal based structure was superior to the combination group and positive control antibody 1394 BMK, as well as superior to the C-terminal-based bi-epitopic antibody Fc-37-46.

### Example 19 Test of the Bi-Epitopic Anti-CD39 Antibody with N-Terminal Based Structure for Reversing T Cell Proliferation Inhibition

By referring to the method described in Example 11, the bi-epitopic anti-CD39 antibody with N-terminal based structure was tested for reversing T cell proliferation inhibition, and the results were shown in FIG. 28.

### Example 20 PK Test of Bi-Epitopic Anti-CD39 Antibody with N-Terminal Based Structure in Mice

Balb/c mice, half male and half female, were maintained at 12/12 light/dark cycle, temperature 24±2° C., humidity 40-70%, with food and water ad libitum. On the day of the experiment, the Balb/c mice were single injected with specific antibody molecule through tail vein at a dose of 10 mg/kg.

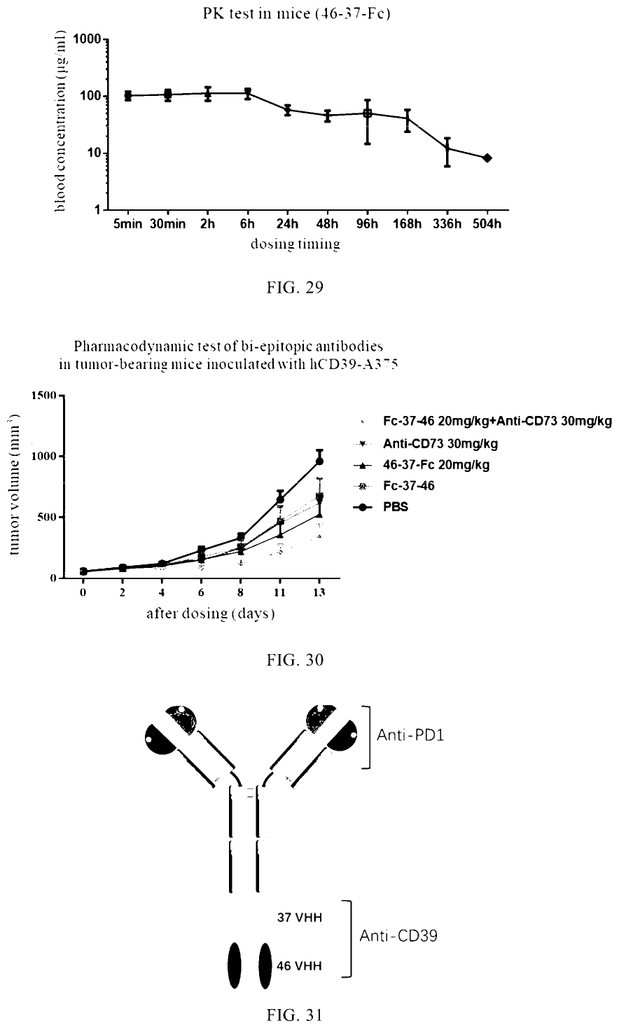

Blood samples were taken from mice orbit at the following timing after dosing: 5 minutes, 0.5 hours, 2 hours, 6 hours, 24 hours, 48 hours, 96 hours, 168 hours, 336 hours, and 504 hours. The whole blood samples were left at 2-8° C. for 30 minutes, followed by centrifugation at 12000 rpm for 5 minutes for serum collection. The obtained serum was then centrifuged at 12000 rpm for 5 minutes at 2-8° C., stored at −80° C. for further determination of blood concentration of anti-CD39 antibody in the serum by ELISA. The results were shown in FIG. 29, indicating that the half-life of 46-37-Fc in mice was 161 hours, and the blood concentration of 46-37-Fc in mice dropped slowly.

### Example 21 Pharmacodynamic Test of Bi-Epitopic Anti-CD39 Antibodies for Anti-Tumor Efficacy in Tumor-Bearing Mice Inoculated with A375 Cell Overexpressing hCD39

In this experiment, anti-CD39 antibodies were tested for anti-tumor efficacy using A375 cells overexpressing hCD39 in B2M KO NDG mice. First, the mice were subcutaneous inoculated with A375 cells overexpressing hCD39, mixed with certain ratio of PBMC cells, to establish the tumor-bearing mice model. When tumors grew to about 50-60 mm³, the mice were injected intraperitoneally with different doses of different antibodies. The mice in each group were monitored for tumor volume and body weight changes at an interval of 2-3 days for 2 weeks. The dosing amounts and administration modes were shown in Table 21. The results were shown in FIG. 30, indicating that, both bi-epitopic anti-CD39 antibodies were anti-tumor effective in this tumor model, the in-vivo anti-tumor effect of 46-37-Fc was superior to that of Fc-37-46; the combination of Fc-37-46 and anti-CD73 antibody (the amino acid sequences of its light and heavy chain were shown in Table 1) resulted in even better in-vivo anti-tumor effect.

TABLE 21
Experimental protocols for tumor inhibition activity

| Group | Dosage | Dosing Times |
|---|---|---|
| Negative control (PBS) | N/A | Q2W*4 |
| 46-37-Fc | 20 mg/kg | Q2W*4 |
| Fc-37-46 | 20 mg/kg | Q2W*4 |
| Anti-CD73 | 30 mg/kg | Q2W*4 |

### Example 22 Construction and Affinity Test of Anti-PD1×CD39 Antibody

Many literatures (e.g., Simoni et al, 2018; Paulino et al, 2021) reported that the co-expression of PD1 and CD39 in tumor infiltrating CD8+ lymphocytes is of great significance for anti-tumor immunity.

Based on the anti-PD1 antibody molecule (ADI-54872, the amino acid sequences of its light and heavy chain variable regions were shown in Table 1), completely independent intellectual property rights owned by BIOTHEUS INC.), an anti-PD1×CD39 antibody is designed to investigate its in-vitro and in-vivo activity. As indicated by the structure shown in FIG. 31, the anti-PD1×CD39 antibody is composed of two peptide chains III-A and two peptide chains III-B, wherein the peptide chain III-A comprises, in order of from N terminal to C terminal: anti-PD-1 antibody VL (SEQ ID NO: 24)-light chain constant region CL (SEQ ID NO: 32), and the peptide chain III-B comprises, in order of from N terminal to C terminal: anti-PD-1 antibody VH (SEQ ID NO: 23)-heavy chain constant region CH1 (SEQ ID NO: 33)-HZ—R-Ye-19 (1)-037-3 VHH (SEQ ID NO: 7)-HZ—R-Ye-19 (1)-046-2 VHH (SEQ ID NO: 16).

### Example 23 Cell Binding Activity of Anti-PD1×CD39 Antibody

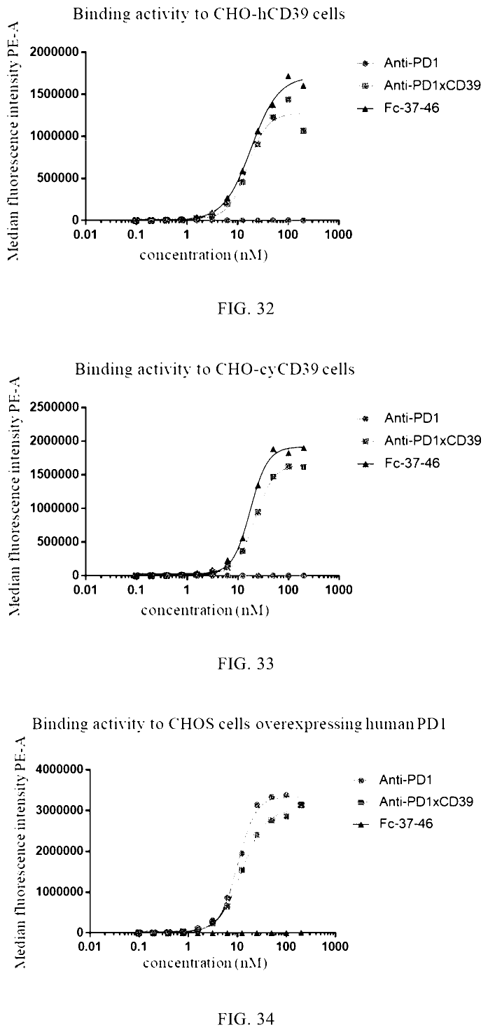

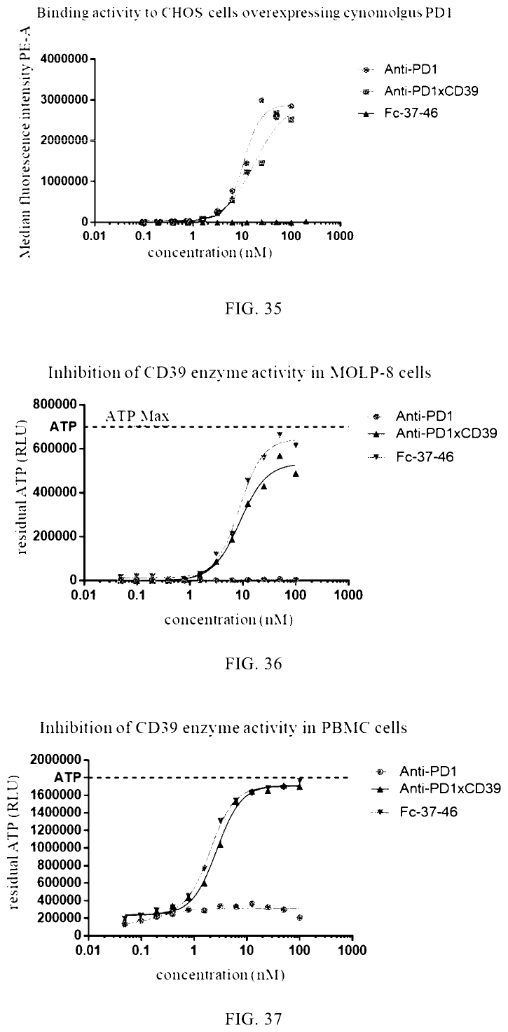

By referring to the experimental method of Example 4, the binding activities of anti-PD1×CD39 antibody to cells overexpressing human CD39, cynomolgus CD39, PD1 (the amino acid sequences of human CD39 and cynomolgus CD39 used were shown respectively in SEQ ID NO: 28 and 29, and the amino acid sequences of human PD-1 and cynomolgus PD-1 used are respectively shown in SEQ ID NO: 30 and 31) were tested respectively. The results were shown in FIGS. 32-35, indicating that the binding activities of the anti-PD1×CD39 antibody to cells overexpressing CD39 or PD1 were consistent with those of the original antibodies.

### Example 24 Functional Activities of the Anti-PD1×CD39 Antibody

By referring to the experimental method of Example 6, the activity of anti-PD1×CD39 antibody for blocking human CD39 enzyme activity was tested first. The results were shown in FIG. 36 and FIG. 37, indicating that the activity of anti-PD1×CD39 antibody for blocking CD39 enzyme activity is comparable to that of the original anti-CD39 antibody molecule.

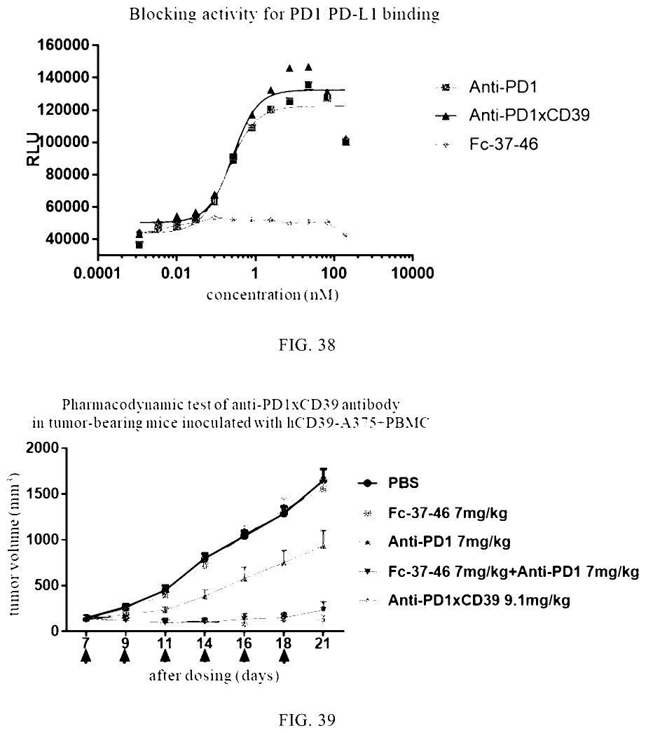

In addition, in order to investigate the activity of anti-PD1×CD39 antibody for blocking PD1, the anti-PD1×CD39 antibody was tested for blocking PD-1/PD-L1 binding, according to the experimental method provided by the PD-1/PD-L1 blockade bioassay (commercially available from Promega, product No: J1250). The activity data were shown in FIG. 38, indicating that the blocking activity of the anti-PD1×CD39 antibody was consistent with the anti-PD-1 monoclonal antibody molecule.

### Example 25 Pharmacodynamic Test of Anti-PD1×CD39 Antibody for Anti-Tumor Efficacy in Tumor-Bearing Mice Inoculated with A375 Cell Overexpressing hCD39

In this experiment, the anti-tumor efficacy were tested using A375 cells overexpressing hCD39 in B2M KO NDG mice. First, the mice were subcutaneous inoculated with A375 cells overexpressing hCD39, mixed with certain ratio of PBMC cells, to establish the tumor-bearing mice model. When tumors grew to about 150-200 mm³, the mice were injected intraperitoneally with different doses of different antibodies. The mice in each group were monitored for tumor volume and body weight changes at an interval of 2-3 days for 2 weeks. The dosing amounts and administration modes were shown in Table 22. The results were shown in FIG. 39, indicating that, in this tumor model, the anti-tumor effect of anti-PD1×CD39 antibody was significantly better than that of monoclonal antibody, and was comparable to or slightly better than that of the combination group.

TABLE 22
Experimental protocols for tumor inhibition activity

| Group | Dosage | | Dosing Times |
|---|---|---|---|
| Negative control (PBS) | N/A | | Q3W*6 |
| Fc-37-46 | 7 | mg/kg | Q3W*6 |
| Anti-PD1 | 7 | mg/kg | Q3W*6 |
| Fc-37-46 + Anti-PD1 | 7 + 7 | mg/kg | Q3W*6 |
| Anti-PD1xCD39 antibody | 9.1 | mg/kg | Q3W*6 |

Although the specific embodiments of the invention have been described in detail, a person skilled in the art will understand that various modifications and changes can be made to the details on the basis of the above disclosure, and such changes are all within the protection scope of the invention. The entire scope of protection of the invention is provided by the appended claims and any equivalents thereof.

What is claimed is:

1. A nanobody or antigen-binding fragment thereof, capable of specifically binding to CD39, comprising:
(a) CDR1, having the sequence shown in SEQ ID NO: 1, or a sequence having substitution, deletion and/or addition of one or more amino acids (e.g., substitution, deletion and/or addition of one, two or three amino acids) as compared with the sequence shown in SEQ ID NO: 1;
(b) CDR2, having the sequence shown in SEQ ID NO: 2, or a sequence having substitution, deletion and/or addition of one or more amino acids (e.g., substitution, deletion and/or addition of one, two or three amino acids) as compared with the sequence shown in SEQ ID NO: 2; and
(c) CDR3, having the sequence shown in SEQ ID NO: 3, or a sequence having substitution, deletion and/or addition of one or more amino acids (e.g., substitution, deletion and/or addition of one, two or three amino acids) as compared with the sequence shown in SEQ ID NO: 3;
preferably, the substitution is a conservative substitution;
preferably, the nanobody or antigen-binding fragment thereof comprises a CDR1 shown in SEQ ID NO: 1, a CDR2 shown in SEQ ID NO: 2, and a CDR3 shown in SEQ ID NO: 3;
preferably, the nanobody or antigen-binding fragment thereof comprises: three CDRs of the VHH as shown in anyone of SEQ ID NOs: 4-8; preferably, the three CDRs of the VHH are determined using Kabat, Chothia or IMGT numbering system.

2. The nanobody or antigen-binding fragment thereof of claim 1, comprising an amino acid sequence selected from:
(i) the sequence shown in SEQ ID NO: 4;
(ii) a sequence having substitution, deletion and or addition of one or more amino acids (e.g., substitution, deletion and or addition of one, two, three, four or five amino acids) as compared with the sequence shown in SEQ ID NO: 4; or
(iii) a sequence having a sequence identity of at least 80%, at least 85%, at least 90%, at least 91%, at least 92%, at least 93%, at least 94%, at least 95%, at least 96%, at least 97%, at least 98%, at least 99%, or 100% with the sequence shown in SEQ ID NO: 4;
preferably, the substitution is a conservative substitution.

3. The nanobody or antigen-binding fragment thereof of claim 1 or 2, wherein the nanobody or antigen-binding fragment thereof is humanized;
preferably, the nanobody or antigen-binding fragment thereof further comprises a heavy chain framework region of human immunoglobulin (e.g., the heavy chain framework region contained in the amino acid sequence encoded by the human heavy chain embryoid antibody gene), and the heavy chain framework region optionally comprises one or more (e.g., 1, 2, 3, 4, 5, 6, 7, 8, 9 or 10) reverse mutations from human residues to camel residues.

4. The nanobody or antigen-binding fragment thereof of claim 3, wherein the nanobody or antigen-binding fragment thereof comprises an amino acid sequence selected from:
(i) the sequence shown in any one of SEQ ID NOs: 5-8;
(ii) a sequence having substitution, deletion and or addition of one or more amino acids (e.g., substitution, deletion and or addition of one, two, three, four or five amino acids) as compared with the sequence shown in any one of SEQ ID NOs: 5-8; or
(iii) a sequence having a sequence identity of at least 80%, at least 85%, at least 90%, at least 91%, at least 92%, at least 93%, at least 94%, at least 95%, at least 96%, at least 97%, at least 98%, at least 99%, or 100% with the sequence shown in any one of SEQ ID NOs: 5-8;
preferably, the substitution is a conservative substitution.

5. A nanobody or antigen-binding fragment thereof, capable of specifically binding to CD39, comprising:
(a) CDR1, having the sequence shown in SEQ ID NO: 9 or 14, or a sequence having substitution, deletion and/or addition of one or more amino acids (e.g., substitution, deletion and/or addition of one, two or three amino acids) as compared with the sequence shown in SEQ ID NO: 9 or 14;
(b) CDR2, having the sequence shown in SEQ ID NO: 10, or a sequence having substitution, deletion and/or addition of one or more amino acids (e.g., substitution, deletion and/or addition of one, two or three amino acids) as compared with the sequence shown in SEQ ID NO: 10; and
(c) CDR3, having the sequence shown in SEQ ID NO: 11 or 15, or a sequence having substitution, deletion and/or addition of one or more amino acids (e.g., substitution, deletion and/or addition of one, two or three amino acids) as compared with the sequence shown in SEQ ID NO: 11 or 15;
preferably, the substitution is a conservative substitution;
preferably, the nanobody or antigen-binding fragment thereof comprises: a CDR1 shown in SEQ ID NO: 9 or 14, a CDR2 shown in SEQ ID NO: 10, and a CDR3 shown in SEQ ID NO: 11 or 15; preferably, the nanobody or antigen-binding fragment thereof comprises:
(1) a CDR1 shown in SEQ ID NO: 9, a CDR2 shown in SEQ ID NO: 10, a CDR3 shown in SEQ ID NO: 11;
(2) a CDR1 shown in SEQ ID NO: 14, a CDR2 shown in SEQ ID NO: 10, a CDR3 shown in SEQ ID NO: 15; or
(3) a CDR1 shown in SEQ ID NO: 14, a CDR2 shown in SEQ ID NO: 10, and a CDR3 shown in SEQ ID NO: 11;
preferably, the nanobody or antigen-binding fragment thereof comprises: three CDRs of the VHH as shown in anyone of SEQ ID NOs: 12, 13 and 16-18; preferably, the three CDRs of the VHH are determined using Kabat, Chothia or IMGT numbering system.

6. The nanobody or antigen-binding fragment thereof of claim 5, comprising an amino acid sequence selected from:
(i) the sequence shown in SEQ ID NO: 12;
(ii) a sequence having substitution, deletion and or addition of one or more amino acids (e.g., substitution, deletion and or addition of one, two, three, four or five amino acids) as compared with the sequence shown in SEQ ID NO: 12; or
(iii) a sequence having a sequence identity of at least 80%, at least 85%, at least 90%, at least 91%, at least 92%, at least 93%, at least 94%, at least 95%, at least 96%, at least 97%, at least 98%, at least 99%, or 100% with sequence shown in SEQ ID NO: 12;
preferably, the substitution is a conservative substitution.

7. The nanobody or antigen-binding fragment thereof of claim 5 or 6, wherein the nanobody or antigen-binding fragment thereof is humanized;
preferably, the nanobody or antigen-binding fragment thereof further comprises a heavy chain framework region of human immunoglobulin (e.g., the heavy chain framework region contained in the amino acid sequence encoded by the human heavy chain embryoid antibody gene), and the heavy chain framework region optionally comprises one or more (e.g., 1, 2, 3, 4, 5, 6, 7, 8, 9 or 10) reverse mutations from human residues to camel residues.

8. The nanobody or antigen-binding fragment thereof of claim 7, wherein the nanobody or antigen-binding fragment thereof comprises an amino acid sequences selected from:
(i) the sequence shown in any one of SEQ ID NOs: 13 and 16-18;
(ii) a sequence having substitution, deletion and or addition of one or more amino acids (e.g., substitution, deletion and or addition of one, two, three, four or five amino acids) as compared with the sequence shown in any one of SEQ ID NOs: 13 and 16-18; or
(iii) a sequence having a sequence identity of at least 80%, at least 85%, at least 90%, at least 91%, at least 92%, at least 93%, at least 94%, at least 95%, at least 96%, at least 97%, at least 98%, at least 99%, or 100% with sequence shown in any one of SEQ ID NOs: 13 and 16-18;
preferably, the substitution is a conservative substitution.

9. The nanobody or antigen-binding fragment thereof of any one of claims 1-8, wherein the CD39 is selected from human CD39 and or cynomolgus CD39;
preferably, the nanobody or antigen-binding fragment thereof can block the enzyme activity of CD39 to which it binds.

10. A polypeptide construct capable of specifically binding to CD39, comprising the nanobody or antigen-binding fragment thereof of any one of claims 1-9, and an immunoglobulin Fc domain;
preferably, the immunoglobulin Fc domain is optionally connected to N terminal and/or C terminal (e.g., C terminal) of the nanobody or antigen-binding fragment thereof through a peptide linker;
preferably, the immunoglobulin Fc domain is a Fc domain of IgG (e.g., Fc domain of IgG1);
preferably, the immunoglobulin Fc domain comprises the sequence shown in SEQ ID NO: 27, or has a sequence identity of at least 80%, at least 85%, at least 90%, at least 91%, at least 92%, at least 93%, at least 94%, at least 95%, at least 96%, at least 97%, at least 98%, at least 99%, or 100% with SEQ ID NO: 27, or a sequence having substitution, deletion and or addition of one or more amino acids (e.g., substitution, deletion and/or addition of one, two, three, four or five amino acids) as compared with the sequence shown in SEQ ID NO: 27.

11. A multi-specific antibody, comprising the nanobody or antigen-binding fragment thereof of any one of claims 1-9 or the polypeptide construct of claim 10;
preferably, the multi-specific antibody is capable of specifically binding to CD39, and additionally is capable of specifically binding to one or more other targets;
preferably, the multi-specific antibody further comprises at least one second antibody having a binding specificity for a second target.

12. A multi-specific antibody, comprising a first antigen-binding domain specific to the first epitope of CD39 and a second antigen-binding domain specific to the second epitope of CD39, wherein the first antigen-binding domain comprises the nanobody or antigen-binding fragment thereof of any one of claims 1-4, and the second antigen-binding domain comprises the nanobody or antigen-binding fragment thereof of any one of claims 5-8.

13. The multi-specific antibody of claim 12, wherein the first antigen-binding domain and the second antigen-binding domain are VHH, and the multi-specific antibody comprises a peptide chain II comprising a monomeric Fc domain, the first antigen-binding domain and the second antigen-binding domain;
preferably, the monomeric Fc domain comprises CH2 and CH3;
preferably, the multi-specific antibody comprises two peptide chains II; preferably, two monomeric Fc domains of the two peptide chains II form a dimer;
preferably, the individual domains are optionally connected by linkers (e.g., a flexible peptide comprising one or more glycine (G) and/or alanine (A));
preferably, the peptide chain II comprises, in order of from N terminal to C terminal, adjacent the first antigen-binding domain and the second antigen-binding domain or adjacent the second antigen-binding domain and the first antigen-binding domain, and further comprises a monomeric Fc domain.

14. The multi-specific antibody of claim 13, wherein the peptide chain II comprises, in order of from N terminal to C terminal, the monomeric Fc domain, the first antigen-binding domain and the second antigen-binding domain;
preferably, the first antigen-binding domain is connected to the C-terminal of the monomeric Fc domain through a linker (e.g., a flexible peptide comprising one or more glycine (G) and/or alanine (A)); and/or, the second antigen-binding domain is connected to the C-terminal of the first antigen-binding domain through a linker (e.g., a flexible peptide comprising one or more glycine (G) and/or alanine (A));
preferably, the first antigen-binding domain comprises or consists of the amino acid sequence shown in SEQ ID NO: 7; and or the second antigen-binding domain comprises or consists of the amino acid sequence shown in SEQ ID NO: 16;
preferably, the peptide chain II comprises or consists of the amino acid sequence shown in SEQ ID NO: 21.

15. The multi-specific antibody of claim 13, wherein the peptide chain II comprises, in order of from N terminal to C terminal, the second antigen-binding domain, the first antigen-binding domain and the monomeric Fc domain;
preferably, the first antigen-binding domain is optionally connected to the C-terminal of the second antigen-binding domain through a linker (e.g., a flexible peptide comprising one or more glycine (G) and/or alanine (A));
preferably, the first antigen-binding domain comprises or consists of the amino acid sequence shown in SEQ ID NO: 7; and or the second antigen-binding domain comprises or consists of the amino acid sequence shown in SEQ ID NO: 16;
preferably, the peptide chain II comprises or consists of the amino acid sequence shown in SEQ ID NO: 22.

16. The multi-specific antibody of claim 12, wherein the first antigen-binding domain and the second antigen-binding domain are VHH, and the multi-specific antibody comprises:
(i) a peptide chain I-A, comprising a first antigen-binding domain and a light chain constant region (CL); and,
(ii) a peptide chain I-B, comprising a second antigen-binding domain and a heavy chain constant region (CH);
preferably, the CL of the peptide chain I-A can form a dimer with the CH1 domain of the heavy chain constant region of the peptide chain I-B;
preferably, the multi-specific antibody comprises two peptide chains I-A and two peptide chains I-B; preferably, the heavy chain constant regions of the two peptide chains I-B form a dimer.

17. The multi-specific antibody of claim 16, wherein:
(1) the peptide chain I-A comprises, in order of from N terminal to C terminal, the first antigen-binding domain and the light chain constant region (CL); and/or,
(2) the peptide chain I-B comprises, in order of from N terminal to C terminal, the second antigen-binding domain and the heavy chain constant region (CH).

18. The multi-specific antibody of claim 16 or 17, wherein:
(i) the first antigen-binding domain comprises or consists of the amino acid sequence shown in SEQ ID NO: 7;
preferably, the peptide chain I-A comprises or consists of the amino acid sequence shown in SEQ ID NO: 20; and/or,
(ii) the second antigen-binding domain comprises or consists of the amino acid sequence shown in SEQ ID NO: 16;
preferably, the peptide chain I-B comprises or consists of the amino acid sequence shown in SEQ ID NO: 19.

19. The multi-specific antibody of any one of claims 12-18, further comprising an antigen-binding domain specific to a target different from CD39.

20. The multi-specific antibody of any one of claims 12-18, further comprises a third antigen-binding domain specific to PD-1.

21. The multi-specific antibody of claim 20, wherein the first antigen-binding domain and the second antigen-binding domain are VHH; the third antigen-binding domain is a Fab, and the multi-specific antibody comprises:
(1) a peptide chain III-A, comprising a light chain variable region of the third antigen-binding domain and a light chain constant region (CL); and,
(2) the peptide chain III-B, comprising a heavy chain variable region of the third antigen-binding domain, a heavy chain constant region, the first antigen-binding domain and the second antigen-binding domain; preferably, the peptide chain III-B comprises, in order of from N terminal to C terminal, adjacent the first antigen-binding domain and the second antigen-binding domain or adjacent the second antigen-binding domain and the first antigen-binding domain, and the peptide chain III-B further comprises the heavy chain variable region of the third antigen-binding domain and the heavy chain constant region;
preferably, the CL of the peptide chain III-A can form a dimer with the CH1 domain of the heavy chain constant region of the peptide chain III-B;
preferably, the multi-specific antibody comprises two peptide chains III-A and two peptide chains III-B; preferably, the heavy chain constant regions of the two peptide chains III-B form a dimer;
preferably, the individual domains are optionally connected therebetween through linkers (e.g., flexible peptides comprising one or more glycine (G) and/or alanine (A)).

22. The multi-specific antibody of claim 21, wherein:
(1) the peptide chain III-A comprises, in order of from N terminal to C terminal, a light chain variable region of the third antigen-binding domain and a light chain constant region (CL); and/or,
(2) the peptide chain III-B comprises, in order of from N terminal to C terminal, the heavy chain variable region of the third antigen-binding domain, the heavy chain constant region, the first antigen-binding domain, and the second antigen-binding domain.

23. The multi-specific antibody of claim 22, having one or more of the following characteristics:
(i) the first antigen-binding domain is connected to the C-terminal of the heavy chain constant region through a linker (e.g., a flexible peptide comprising one or more glycine (G) and/or alanine (A)); and/or, the second antigen-binding domain is connected to the C-terminal of the first antigen-binding domain through a linker (e.g., a flexible peptide comprising one or more glycine (G) and/or alanine (A));
(ii) the first antigen-binding domain comprises or consists of the amino acid sequence shown in SEQ ID NO: 7;
(iii) the second antigen-binding domain comprises or consists of the amino acid sequence shown in SEQ ID NO: 16;
(iv) the heavy chain variable region of the third antigen-binding domain comprises VH CDRs1-3 set forth in SEQ ID NOs: 36-38 respectively;
(v) the light chain variable region of the third antigen-binding domain comprises VL CDRs 1-3 set forth in SEQ ID NOs: 39-41 respectively;
(vi) the heavy chain variable region of the third antigen-binding domain comprises or consists of the amino acid sequence shown in SEQ ID NO: 23;
(vii) the light chain variable region of the third antigen-binding domain comprises or consists of the amino acid sequence shown in SEQ ID NO: 24;
preferably, the peptide chain III-A comprises or consists of the amino acid sequence shown in SEQ ID NO: 26;
preferably, the peptide chain III-B comprises or consists of the amino acid sequence shown in SEQ ID NO: 25.

24. A multi-specific antibody capable of specifically binding to both CD39 and PD-1, comprising the nanobody or antigen-binding fragment thereof of any one of claims 1-9 or the polypeptide construct of claim 10, and an antigen-binding domain specific to PD-1;
preferably, the antigen-binding domain specific to PD-1 comprises VH CDRs 1-3 set forth in SEQ ID NOs: 36-38 respectively, and/or VL CDRs 1-3 set forth in SEQ ID NOs: 39-41 respectively;
preferably, the antigen-binding domain specific to PD-1 comprises a VH shown in SEQ ID NO: 23, and/or a VL shown in SEQ ID NO: 24.

25. An isolated nucleic acid molecule, encoding the nanobody or antigen-binding fragment thereof of any one of claims 1-9, the polypeptide construct of claim 10, or the multi-specific antibody of any one of claims 11-24.

26. A vector, comprising the isolated nucleic acid molecule of claim 25; preferably, the vector is a cloning vector or an expression vector.

27. A host cell, comprising the nucleic acid molecule of claim 25 or the vector of claim 26.

28. A method for preparing the nanobody or antigen-binding fragment thereof of any one of claims 1-9, the polypeptide construct of claim 10, or the multi-specific antibody of any one of claims 11-24, the method comprises cultivating the host cell of claim 27 under the condition of allowing protein expression, and recovering the nanobody or antigen-binding fragment thereof or the polypeptide construct or the multi-specific antibody from the cultured host cell culture.

29. A composition, comprising:
(i) the nanobody or antigen-binding fragment thereof of any one of claims 1-4, a polypeptide construct comprising the nanobody or antigen-binding fragment thereof, a nucleic acid molecule encoding the nanobody or antigen-binding fragment thereof, a vector comprising the nucleic acid molecule, or a host cell comprising the nucleic acid molecule or vector; and
(ii) the nanobody or antigen-binding fragment thereof of any one of claims 5-8, a polypeptide construct comprising the nanobody or antigen-binding fragment thereof, a nucleic acid molecule encoding the nanobody or antigen-binding fragment thereof, a vector comprising the nucleic acid molecule, or a host cell comprising the nucleic acid molecule or vector.

30. A pharmaceutical composition, comprising the nanobody or antigen-binding fragment thereof of any one of claims 1-9, or the polypeptide construct of claim 10, or the multi-specific antibody of any one of claims 11-24, or the isolated nucleic acid molecule of claim 25, or the vector of claim 26, or the host cell of claim 27, or the composition of claim 29, and pharmaceutically acceptable carrier and/or excipient;
preferably, the pharmaceutical composition further comprises an immune checkpoint inhibitor;
preferably, the immune checkpoint inhibitor is selected from anti-PD-1 antibody, anti-PD-L1 antibody, anti-CD73 antibody, or a combination thereof;
preferably, the anti-PD-1 antibody comprises VH CDRs 1-3 set forth in SEQ ID NOs: 36-38 respectively, and/or VL CDRs 1-3 set forth in SEQ ID NOs: 39-41 respectively;
preferably, the anti-PD-1 antibody comprises a VH shown in SEQ ID NO: 23, and/or a VL shown in SEQ ID NO: 24.

31. Use of the nanobody or antigen-binding fragment thereof of any one of claims 1-9, or the polypeptide construct of claim 10, or the multi-specific antibody of any one of claims 11-24, or the isolated nucleic acid molecule of claim 25, or the vector of claim 26, or the host cell of claim 27, or the composition of claim 29, in the preparation of a medicament for:
(1) reducing the enzyme activity of CD39 in-vitro or in-vivo (e.g., in a human);
(2) alleviating adenosine mediated immunosuppression in a subjuect (e.g., human);
(3) preventing and/or treating tumors in a subject (e.g., human); or
(4) preventing and/or treating infection in a subject (e.g., human);
preferably, the tumor involves CD39 positive tumor cells;
preferably, the tumor is selected from solid tumor or blood tumor (e.g., leukemia, lymphoma);
preferably, the tumor is selected from colorectal cancer, colon cancer, bladder cancer, breast cancer, uterine/cervical cancer, ovarian cancer, prostate cancer, testicular cancer, esophageal cancer, gastrointestinal cancer, pancreatic cancer, kidney cancer, head and neck cancer, lung cancer, stomach cancer, germ cell cancer, bone cancer, liver cancer, thyroid carcinoma, skin cancer, tumor of the central nervous system, lymphoma, leukemia, myeloma, sarcoma, and melanoma;
preferably, the infection is selected from viral infection, bacterial infection, fungal infection, and parasitic infection;
preferably, the subject is a mammal, such as, a human or a monkey;
preferably, the nanobody or antigen-binding fragment thereof, or the polypeptide construct, or the multi-specific antibody, or the isolated nucleic acid molecule, or the vector, or the host cell, or the composition is used alone, or in combination with other pharmaceutical active agent(s);
preferably, the nanobody or antigen-binding fragment thereof, or the polypeptide construct, or the multi-specific antibody, or the isolated nucleic acid molecule, or the vector, or the host cell, or the composition is used in combination with an immune checkpoint inhibitor;
preferably, the immune checkpoint inhibitor is selected from anti-PD-1 antibody, anti-PD-L1 antibody, anti-CD73 antibody, or a combination thereof.

32. A method for enhancing immune response or preventing and/or treating tumor or infection in a subject, comprising: administering an effective amount of the nanobody or antigen-binding fragment thereof of any one of claims 1-9, or the peptide construct of claim 10, or the multi-specific antibody of any one of claims 11-24, or the isolated nucleic acid molecule of claim 25, or the vector of claim 26, or the host cell of claim 27, or the composition of claim 29, or the pharmaceutical composition of claim 30, to the subject in need thereof;
preferably, the tumor involves CD39 positive tumor cells;
preferably, the tumor is selected from solid tumor or blood tumor (e.g., leukemia, lymphoma);
preferably, the tumor is selected from colorectal cancer, colon cancer, bladder cancer, breast cancer, uterine/cervical cancer, ovarian cancer, prostate cancer, testicular cancer, esophageal cancer, gastrointestinal cancer, pancreatic cancer, kidney cancer, head and neck cancer, lung cancer, stomach cancer, germ cell cancer, bone cancer, liver cancer, thyroid carcinoma, skin cancer, tumor of the central nervous system, lymphoma, leukemia, myeloma, sarcoma, and melanoma;
preferably, the infection is selected from viral infection, bacterial infection, fungal infection, and parasitic infection;
preferably, the subject is a mammal, such as, a human or a monkey;
preferably, the nanobody or antigen-binding fragment thereof, or the polypeptide construct, or the multi-specific antibody, or the isolated nucleic acid molecule, or the vector, or the host cell, or the composition is administered alone, or in combination with other pharmaceutical active agent(s);
preferably, the nanobody or antigen-binding fragment thereof, or the polypeptide construct, or the multi-specific antibody, or the isolated nucleic acid molecule, or the vector, or the host cell, or the combination is administered in combination with an immune checkpoint inhibitor;
preferably, the immune checkpoint inhibitor is selected from anti-PD-1 antibody, anti-PD-L1 antibody, anti-CD73 antibody, or a combination thereof.

33. A conjugate, comprising the nanobody or antigen-binding fragment thereof of any one of claims 1-9, or the polypeptide construct of claim 10, and a detectable marker connected with the nanobody or antigen-binding fragment thereof or the polypeptide construct;
preferably, the detectable marker is selected from an enzyme (e.g., horseradish peroxidase or alkaline phosphatase), a chemiluminescence reagent (e.g., acridine ester compound, luminol and its derivative, or ruthenium derivative), a fluorescent dye (e.g., fluorescein or fluorescent protein), a radionuclide or biotin.

34. A kit, comprising the nanobody or antigen-binding fragment thereof of any one of claims 1-9, or the polypeptide construct of claim 10, or the conjugate of claim 33;
preferably, the kit comprises the conjugate of claim 33;
preferably, the kit comprises the nanobody or antigen-binding fragment thereof of any one of claims 1-9 or the polypeptide construct of claim 10, and a second antibody capable of specifically recognizing the nanobody or antigen-binding fragment thereof; optionally, the second antibody further comprises a detectable marker, such as an enzyme (e.g., horseradish peroxidase or alkaline phosphatase), a chemiluminescence reagent (e.g., acridine ester compound, luminol and its derivative, or ruthenium derivative), a fluorescent dye (e.g., fluorescein or fluorescent protein), a radionuclide or biotin.

35. A method for detecting the presence or level of CD39 in a sample, comprising the use of the nanobody or antigen-binding fragment thereof of any one of claims 1-9, the polypeptide construct of claim 10, or the conjugate of claim 33;
preferably, the method is an immunoassay, such as immunoblotting assay, enzyme immunoassay (e.g., ELISA), chemiluminescence immunoassay, fluoroimmunoassay, or radioimmunoassay;
preferably, the method comprises using the conjugate of claim 33;
preferably, the method comprises using the nanobody or antigen-binding fragment thereof of any one of claims 1-9 or the polypeptide construct of claim 10, and the method further comprises using a second antibody carrying a detectable marker (such as, an enzyme (e.g., horseradish peroxidase or alkaline phosphatase), a chemiluminescence reagent (e.g., acridine ester compound, luminol and its derivative, or ruthenium derivative), a fluorescent dye (e.g., fluorescein or fluorescent protein), a radionuclide or biotin), for detecting the nanobody or antigen-binding fragment thereof or the polypeptide construct.

36. The method of claim 35, comprising:
(1) contacting the sample with the nanobody or antigen-binding fragment thereof of any one of claims 1-9, the polypeptide construct of claim 10, or the conjugate of claim 33;
(2) detecting the formation of antigen-antibody immune complex or determining the amount of the immune complex, wherein the formation of the immune complex indicates the presence of CD39 or CD39-expressing cells.

37. Use of the nanobody or antigen-binding fragment thereof of any one of claims 1-9, the polypeptide construct of claim 10, or the conjugate of claim 33, in the preparation of a test reagent for detecting the presence or level of CD39 in a sample;
preferably, the test reagent is used for detecting the presence or level of CD39 in the sample by the method of claim 35 or 36;
preferably, the sample is a cell sample (e.g., tumor cell) derived from a subject (e.g., a mammal, preferably a human or monkey).


SEQUENCE LISTING (43 sequences)

SEQ ID NO: 1 - protein, 5 aa, synthetic construct
  source 1..5: note = R-Ye-19(1)-037/HZ-R-Ye-19(1)-037-1/HZ-R-Ye-19(1)-03 7-2/ HZ-R-Ye-19(1)-037-3/HZ-R-Ye-19(1)-037-4 CDR1
EYSMG

SEQ ID NO: 2 - protein, 17 aa, synthetic construct
  source 1..17: note = R-Ye-19(1)-037/HZ-R-Ye-19(1)-037-1/HZ-R-Ye-19(1)-03 7-2/ HZ-R-Ye-19(1)-037-3/HZ-R-Ye-19(1)-037-4 CDR2
AISGFGHITHYADSVKG

SEQ ID NO: 3 - protein, 17 aa, synthetic construct
  source 1..17: note = R-Ye-19(1)-037/HZ-R-Ye-19(1)-037-1/HZ-R-Ye-19(1)-03 7-2/ HZ-R-Ye-19(1)-037-3/HZ-R-Ye-19(1)-037-4 CDR3
AWQTSPRRMMQMAGSIT

SEQ ID NO: 4 - protein, 126 aa, synthetic construct
  source 1..126: note = R-Ye-19(1)-037 VHH
QVQLQESGGGLVQAGGSLRLACTVSRGTFSEYSMGWFRQAPGQERDFVTAISGFGHITHYADSVKGRFTISRDNAANTVYLQMNTLKPEDTAVYYCAAAWQTSPRRMMQMAGSITWGQGTQVTVSS

SEQ ID NO: 5 - protein, 126 aa, synthetic construct
  source 1..126: note = HZ-R-Ye-19(1)-037-1 VHH
EVQLLESGGGLVQPGGSLRLSCTVSRGTFSEYSMGWFRQAPGQERDFVTAISGFGHITHYADSVKGRFTISRDNSKNTVYLQMNSLKPEDTAVYYCAAAWQTSPRRMMQMAGSITWGQGTQVTVSS

SEQ ID NO: 6 - protein, 126 aa, synthetic construct
  source 1..126: note = HZ-R-Ye-19(1)-037-2 VHH
EVQLLESGGGLVQPGGSLRLSCAVSRGTFSEYSMSWFRQAPGQERDFVTAISGFGHITHYADSVKGRFTISRDNSKNTVYLQMNSLRAEDTAVYYCAAAWQTSPRRMMQMAGSITWGQGTQVTVSS

SEQ ID NO: 7 - protein, 126 aa, synthetic construct
  source 1..126: note = HZ-R-Ye-19(1)-037-3 VHH
EVQLLESGGGLVQPGGSLRLSCAVSRGTFSEYSMSWFRQAPGKEREFVTAISGFGHITHYADSVKGRFTISRDNSKNTVYLQMNSLRAEDTAVYYCAAAWQTSPRRMMQMAGSITWGQGTQVTVSS

SEQ ID NO: 8 - protein, 126 aa, synthetic construct
  source 1..126: note = HZ-R-Ye-19(1)-037-4 VHH
EVQLLESGGGLVQPGGSLRLSCAVSRGTFSEYSMSWFRQAPGKGLEFVTAISGFGHITHYADSVKGRFTISRDNSKNTVYLQMNSLRAEDTAVYYCAAAWQTSPRRMMQMAGSITWGQGTQVTVSS

SEQ ID NO: 9 - protein, 5 aa, synthetic construct
  source 1..5: note = R-Ye-19(1)-046/HZ-R-Ye-19(1)-046-1 CDR1
INYMG

SEQ ID NO: 10 - protein, 16 aa, synthetic construct
  source 1..16: note = R-Ye-19(1)-046/HZ-R-Ye-19(1)-046-1/HZ-R-Ye-19(1)-04 6-2/ HZ-R-Ye-19(1)-046-3/HZ-R-Ye-19(1)-046-4 CDR2
AITGRGNTNYADSVKG

SEQ ID NO: 11 - protein, 16 aa, synthetic construct
  source 1..16: note = R-Ye-19(1)-046/HZ-R-Ye-19(1)-046-1/HZ-R-Ye-19(1)-04 6-3/ HZ-R-Ye-19(1)-046-4 CDR3
WDYSDYDEGPLREYDY

SEQ ID NO: 12 - protein, 124 aa, synthetic construct
  source 1..124: note = R-Ye-19(1)-046 VHH
QVQLQESGGGLVQTGRSLRLSCAASGDIFSINYMGWYRQAPGNERELVAAITGRGNTNYADSVKGRFTISRDNAKNTVYLQMNSLKPEDTAVYYCNAWDYSDYDEGPLREYDYWGQGTQVTVSS

SEQ ID NO: 13 - protein, 124 aa, synthetic construct
  source 1..124: note = HZ-R-Ye-19(1)-046-1 VHH
EVQLLESGGGLVQPGRSLRLSCAASGDIFSINYMGWYRQAPGNERELVAAITGRGNTNYADSVKGRFTISRDNSKNTVYLQMNSLKPEDTAVYYCNAWDYSDYDEGPLREYDYWGQGTQVTVSS

SEQ ID NO: 14 - protein, 5 aa, synthetic construct
  source 1..5: note = HZ-R-Ye-19(1)-046-2/HZ-R-Ye-19(1)-046-3/ HZ-R-Ye-19(1)-046-4 CDR1
INYMS

SEQ ID NO: 15 - protein, 16 aa, synthetic construct
  source 1..16: note = HZ-R-Ye-19(1)-046-2 CDR3
WDYSDADEGPLREYDY

SEQ ID NO: 16 - protein, 124 aa, synthetic construct
  source 1..124: note = HZ-R-Ye-19(1)-046-2 VHH
EVQLLESGGGLVQPGGSLRLSCAASGDIFSINYMSWYRQAPGNERELVAAITGRGNTNYADSVKGRFTISRDNSKNTVYLQMNSLRAEDTAVYYCNAWDYSDADEGPLREYDYWGQGTQVTVSS

SEQ ID NO: 17 - protein, 124 aa, synthetic construct
  source 1..124: note = HZ-R-Ye-19(1)-046-3 VHH
EVQLLESGGGLVQPGGSLRLSCAASGDIFSINYMSWYRQAPGKERELVAAITGRGNTNYADSVKGRFTISRDNSKNTVYLQMNSLRAEDTAVYYCNAWDYSDYDEGPLREYDYWGQGTQVTVSS

SEQ ID NO: 18 - protein, 124 aa, synthetic construct
  source 1..124: note = HZ-R-Ye-19(1)-046-4 VHH
EVQLLESGGGLVQPGGSLRLSCAASGDIFSINYMSWYRQAPGKGLELVAAITGRGNTNYADSVKGRFTISRDNSKNTVYLQMNSLRAEDTAVYYCNAWDYSDYDEGPLREYDYWGQGTQVTVSS

SEQ ID NO: 19 - protein, 453 aa, synthetic construct
  source 1..453: note = Bi307/308-H
EVQLLESGGGLVQPGGSLRLSCAASGDIFSINYMSWYRQAPGNERELVAAITGRGNTNYADSVKGRFTISRDNSKNTVYLQMNSLRAEDTAVYYCNAWDYSDADEGPLREYDYWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 233 aa, synthetic construct
  source 1..233: note = Bi307/308-L
EVQLLESGGGLVQPGGSLRLSCAVSRGTFSEYSMSWFRQAPGKEREFVTAISGFGHITHYADSVKGRFTISRDNSKNTVYLQMNSLRAEDTAVYYCAAAWQTSPRRMMQMAGSITWGQGTQVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 532 aa, synthetic construct
  source 1..532: note = Fc-37-46
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGAGGGGAGGGGAGGGGAEVQLLESGGGLVQPGGSLRLSCAVSRGTFSEYSMSWFRQAPGKEREFVTAISGFGHITHYADSVKGRFTISRDNSKNTVYLQMNSLRAEDTAVYYCAAAWQTSPRRMMQMAGSITWGQGTQVTVSSGGGGAGGGGAGGGGAGGGGAGGGGAGGGGAGGGGAEVQLLESGGGLVQPGGSLRLSCAASGDIFSINYMSWYRQAPGNERELVAAITGRGNTNYADSVKGRFTISRDNSKNTVYLQMNSLRAEDTAVYYCNAWDYSDADEGPLREYDYWGQGTQVTVSSA

SEQ ID NO: 22 - protein, 496 aa, synthetic construct
  source 1..496: note = 46-37-Fc
EVQLLESGGGLVQPGGSLRLSCAASGDIFSINYMSWYRQAPGNERELVAAITGRGNTNYADSVKGRFTISRDNSKNTVYLQMNSLRAEDTAVYYCNAWDYSDADEGPLREYDYWGQGTQVTVSSGGGGAGGGGAGGGGAGGGGAEVQLLESGGGLVQPGGSLRLSCAVSRGTFSEYSMSWFRQAPGKEREFVTAISGFGHITHYADSVKGRFTISRDNSKNTVYLQMNSLRAEDTAVYYCAAAWQTSPRRMMQMAGSITWGQGTQVTVSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 120 aa, synthetic construct
  source 1..120: note = VH of anti-PD1 antibody
EVQLVQSGAEVKKPGASVKVSCKASGYTFTEYYIYWVRQAPGQGLEWIGGINPSNGGTNFNEKFKPRVTMTVDTSTSTAYMELSSLRSEDTAVYYCTVRDFRFDKGFKYWGQGTLVTVSS

SEQ ID NO: 24 - protein, 111 aa, synthetic construct
  source 1..111: note = VL of anti-PD1 antibody
DIVLTQSPATLSLSPGERATLSCRASKSVSTSGLNYVHWYQRKPGQAPRLLIYLGSYLDSGVPARFSGSGSGTDFTLTISSLEPEDFAVYYCQQSWELPLTFGGGTKVEIK

SEQ ID NO: 25 - protein, 755 aa, synthetic construct
  source 1..755: note = Heavy chain of anti- PD1xCD39 antibody
EVQLVQSGAEVKKPGASVKVSCKASGYTFTEYYIYWVRQAPGQGLEWIGGINPSNGGTNFNEKFKPRVTMTVDTSTSTAYMELSSLRSEDTAVYYCTVRDFRFDKGFKYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGAGGGGAGGGGAGGGGAEVQLLESGGGLVQPGGSLRLSCAVSRGTFSEYSMSWFRQAPGKEREFVTAISGFGHITHYADSVKGRFTISRDNSKNTVYLQMNSLRAEDTAVYYCAAAWQTSPRRMMQMAGSITWGQGTQVTVSSGGGGAGGGGAGGGGAGGGGAGGGGAGGGGAGGGGAEVQLLESGGGLVQPGGSLRLSCAASGDIFSINYMSWYRQAPGNERELVAAITGRGNTNYADSVKGRFTISRDNSKNTVYLQMNSLRAEDTAVYYCNAWDYSDADEGPLREYDYWGQGTQVTVSSA

SEQ ID NO: 26 - protein, 218 aa, synthetic construct
  source 1..218: note = Light chain of anti-PD1xCD39 antibody
DIVLTQSPATLSLSPGERATLSCRASKSVSTSGLNYVHWYQRKPGQAPRLLIYLGSYLDSGVPARFSGSGSGTDFTLTISSLEPEDFAVYYCQQSWELPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 226 aa, synthetic construct
  source 1..226: note = human IgG1 Fc region
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 510 aa, synthetic construct
  source 1..510: note = human CD39
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 29 - protein, 522 aa, synthetic construct
  source 1..522: note = cynomolgus CD39
MGREELYLTFSFSSGFRESNVKTFCSKNILAILGFSSIIAVIALLAVGLTQNKALPENIKYGIVLDAGSSHTSLYIYKWPAEKENDTGVVHQVEECRVKGPGISKYVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQITFVPQNQTTESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSVLELFNTSYCPYSQCAFNGIFLPPLQGDFGAFSAFYFVMNFLNLTSEKVSQEKVTEMMKKFCSQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLVIVAIIGLLIFHKPSYFWKDMV

SEQ ID NO: 30 - protein, 288 aa, synthetic construct
  source 1..288: note = Human PD-1
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 31 - protein, 288 aa, synthetic construct
  source 1..288: note = Cynomolgus PD-1
MQIPQAPWPVVWAVLQLGWRPGWFLESPDRPWNPPTFSPALLLVTEGDNATFTCSFSNASESFVLNWYRMSPSNQTDKLAAFPEDRSQPGRDCRFRVTQLPNGRDFHMSVVRARRNDSGTYLCGAISLAPKAQIKESLRAELRVTERRAEVPTAHPSPSPRPAGQFQALVVGVVGGLLGSLVLLVWVLAVICSRAAQGTIEARRTGQPLKEDPSAVPVFSVDYGELDFQWREKTPEPPAPCVPEQTEYATIVFPSGLGTSSPARRGSADGPRSPRPLRPEDGHCSWPL

SEQ ID NO: 32 - protein, 107 aa, synthetic construct [sequence omitted]

SEQ ID NO: 33 - protein, 329 aa, synthetic construct
  source 1..329: note = heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 445 aa, synthetic construct
  source 1..445: note = I394 BMK heavy chain
EVQLQQSGPELVKPGASVKMSCKASGYTFTDYNMHWVKQSHGRTLEWIGYIVPLNGGSTFNQKFKGRATLTVNTSSRTAYMELRSLTSEDSAAYYCARGGTRFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 218 aa, synthetic construct
  source 1..218: note = I394 BMK light chain
DIVLTQSPASLAVSLGQRATISCRASESVDNFGVSFMYWFQQKPGQPPNLLIYGASNQGSGVPARFRGSGSGTDFSLNIHPMEADDTAMYFCQQTKEVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 5 aa, synthetic construct
  source 1..5: note = HCDR1 of anti-PD1 antibody
EYYIY

SEQ ID NO: 37 - protein, 17 aa, synthetic construct
  source 1..17: note = HCDR2 of anti-PD1 antibody
GINPSNGGTNFNEKFKP

SEQ ID NO: 38 - protein, 11 aa, synthetic construct
  source 1..11: note = HCDR3 of anti-PD1 antibody
RDFRFDKGFKY

SEQ ID NO: 39 - protein, 15 aa, synthetic construct
  source 1..15: note = LCDR1 of anti-PD1 antibody
RASKSVSTSGLNYVH

SEQ ID NO: 40 - protein, 7 aa, synthetic construct
  source 1..7: note = LCDR2 of anti-PD1 antibody
LGSYLDS

SEQ ID NO: 41 - protein, 9 aa, synthetic construct
  source 1..9: note = LCDR3 of anti-PD1 antibody
QQSWELPLT

SEQ ID NO: 42 - protein, 447 aa, synthetic construct
  source 1..447: note = Heavy chain of anti-CD73 antibody
QVQLQESGPGLVKPSQTLSLTCTVSGYSITSGYDWHWIRQHPGKGLEWMGYISYSGYTDYNPSLKSRITISHDTSKNQFSLKLSSVTAADTAVYYCTRGDHSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 214 aa, synthetic construct
  source 1..214: note = Light chain of anti-CD73 antibody
DIQMTQSHSSLSASVGDRVTITCKASQAVGTAVAWYQQKPGKSPKLLIYWASSRHTGVPSRFSGSRSGTDFTLTISSLQPEDFATYFCQQYSSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC